SEQUENCE LISTING (1 sequence)

SEQ ID NO: 1 - protein, 385 aa, Bacillus subtilis
MKKQNDIPQPIRGDKGATVKIPRNIERDRQNPDMLVPPETDHGTVSNMKFSFSDTHNRLEKGGYAREVTVRELPISENLASVNMRLKPGAIRELHWHKEAEWAYMIYGSARVTIVDEKGRSFIDDVGEGDLWYFPSGLPHSIQALEEGAEFLLVFDDGSFSENSTFQLTDWLAHTPKEVLAANFGVTKEEISNLPGKEKYIFENQLPGSLKDDLVEGPNGEVPYPETYRLLEQEPIESEGGKVYIADSTNFKVSKTIASALVTVEPGAMRELHWHPNTHEWQYYISGKARMTVFASDGHARTFNYQAGDVGYVPFAMGHYVENIGDEPLVFLEIFKDDHYADVSLNQWLAMLPETFVQAHLDLGKDFTDVLSKEKHPVVKKKCSK

(19) United States
(12) Patent Application Publication
(10) Pub. No.: US 2022/0378886 A1
(43) Pub. Date: Dec. 1, 2022

(54) METHODS OF TREATING HYPEROXALURIA

(71) Applicant: Allena Pharmaceuticals, Inc., Newton, MA (US)

(72) Inventors: Danica Grujic, Boston, MA (US); Kristine E. Bernard, Newton, MA (US); Annamaria T. Kausz, Newton, MA (US); Zhiqun Zhang, Newton, MA (US)

(21) Appl. No.: 17/774,974

(22) PCT Filed: Nov. 6, 2020
(86) PCT No.: PCT/US2020/059520

§ 371 Date: May 6, 2022

Related U.S. Application Data
(60) Provisional application No. 62/931,771, filed on Nov. 6, 2019.

(51) Int. Cl. A61K 38/51; A61P 1/00; A61K 9/00

(52) U.S. Cl. CPC A61K 38/51; C12Y 401/01002; A61P 1/00; A61K 9/0053

(57) ABSTRACT

Disclosed are methods and compositions which can be used to treat diseases or disorders associated with an elevated amount of oxalate, including, for example, hyperoxaluria, in particular enteric hyperoxaluria.

### CROSS REFERENCE TO RELATED APPLICATIONS

This application claims the benefit of, and priority to, U.S. Provisional Patent Application No. 62/931,771, filed on Nov. 6, 2019, which is hereby incorporated by reference herein in its entirety.

### SEQUENCE LISTING

The instant application contains a Sequence Listing which has been submitted electronically in ASCII format and is hereby incorporated by reference in its entirety. Said ASCII copy, created on Nov. 6, 2020, is named ALE-008WO-SL.txt and is 3.51 kb in size.

### FIELD OF THE INVENTION

The invention relates generally to methods and compositions for treating diseases or disorders associated with an elevated amount of oxalate, e.g., hyperoxaluria.

### BACKGROUND

Hyperoxaluria is a metabolic disorder characterized by significantly elevated oxalate levels in the urine, or urinary oxalate excretion, due to either overproduction of oxalate by the liver caused by a genetic defect, called primary hyperoxaluria, or from the excess absorption of oxalate from the diet, called secondary hyperoxaluria. Secondary hyperoxaluria is further characterized either as enteric, resulting from a chronic and unremediable underlying gastrointestinal disorder associated with malabsorption, such as bariatric surgery complications or Crohn's disease, which predisposes subjects to excess oxalate absorption, or idiopathic, meaning the underlying cause is unknown. Kidney stones, typically the first sign of hyperoxaluria, are often painful and may require interventional procedures. Severe hyperoxaluria associated with enteric or primary hyperoxaluria may also lead to kidney damage (nephrocalcinosis), chronic kidney disease and end-stage renal disease, which may lead to death.

Enteric hyperoxaluria is the more severe subset of secondary hyperoxaluria. It is estimated that there are approximately 200,000 to 250,000 subjects with enteric hyperoxaluria and kidney stones in the United States.

Although developments have been made to date, there is still an ongoing need for new and effective therapies for treating and managing diseases or disorders associated with an elevated amount of oxalate such as hyperoxaluria.

### SUMMARY OF THE INVENTION

The invention is based, in part, upon the discovery of a dosing regimen for treating a subject with enteric hyperoxaluria or treating a subject with enteric hyperoxaluria and at risk of developing or with advanced chronic kidney disease (CKD) with an effective amount of biologically active oxalate decarboxylase (OXDC) crystals up to 5 times per day, wherein when the dosing regimen is administered to a subject with enteric hyperoxaluria or a subject with enteric hyperoxaluria and at risk of developing or with advanced chronic kidney disease (CKD), the dosing regimen causes significant reductions in the baseline level of 24-hour urinary oxalate (UOx) excretion and/or plasma oxalate (POx).

In one aspect, the invention provides a method of treating a subject with having enteric hyperoxaluria, the method comprising orally administering to the subject an effective amount of biologically active oxalate decarboxylase (OXDC) crystals up to 5 times per day; wherein the level of 24-hour urinary oxalate (UOx) excretion of the subject is reduced by at least 20% relative to the level of 24-hour UOx excretion prior to treatment. In certain embodiments, the subject (i) is receiving a proton pump inhibitor and/or an acid blocker, and/or (ii) is at risk of developing or has advanced chronic kidney disease (CKD).

In certain embodiments, the subject (i) has had bariatric surgery, (ii) is receiving a proton pump inhibitor and/or an acid blocker, or (iii) has had bariatric surgery and is receiving a proton pump inhibitor and/or an acid blocker.

In one aspect, the invention provides a method of treating a subject with enteric hyperoxaluria, the method comprising administering a dosing regimen of biologically active oxalate decarboxylase (OXDC) crystals to the subject, wherein, when the dosing regimen is administered to such subjects with enteric hyperoxaluria, the dosing regimen causes about a 20% mean reduction in the baseline level of 24-hour UOx excretion. In certain embodiments, when the dosing regimen is administered to such a subject with enteric hyperoxaluria, the dosing regimen causes a reduction in kidney stone disease progression in the subject. For example, when the dosing regimen is administered to such subjects, the dosing regimen causes a reduction in kidney stone disease progression in at least about 5%, 10%, 15%, 20%, 25%, 30%, 35%, or 40% of the subjects and/or a reduction in kidney stone disease progression in a proportion of subjects that is at least about 5%, 10%, 15%, 20%, 25%, 30%, 35%, or 40% greater than the proportion of untreated subjects with a reduction in kidney stone disease progression.

In one aspect, the invention provides a method of treating a subject having enteric hyperoxaluria, the method comprising orally administering to the subject an effective amount of biologically active oxalate decarboxylase (OXDC) crystals up to 5 times per day; wherein the subject (i) has had bariatric surgery, (ii) is receiving a proton pump inhibitor and/or an acid blocker, or (iii) has had bariatric surgery and is receiving a proton pump inhibitor and/or an acid blocker.

In certain embodiments, the level of 24-hour UOx excretion of the subject is reduced by at least 20% relative to the level of 24-hour UOx excretion prior to treatment.

In certain embodiments, the OXDC crystals are administered every day for at least 28 days. In certain embodiments, the OXDC crystals reduce 24-hour UOx excretion within 7 days after the initial administration of the OXDC crystals.

In one aspect, the invention provides a method of treating a subject with enteric hyperoxaluria and advanced chronic kidney disease (CKD), the method comprising orally administering to the subject an effective amount of biologically active oxalate decarboxylase (OXDC) crystals up to 5 times per day, whereupon administration of the OXDC crystals causes (a) a reduction in the level of 24-hour urinary oxalate (UOx) excretion of the subject by 25-50% relative to the level of 24-hour UOx excretion prior to treatment; and/or (b) a reduction in the plasma oxalate (POx) level of the subject by 15-80% relative to the level of POx prior to treatment.

In certain embodiments, the subject in need thereof has (i) UOx excretion of >40 mg/24 hours (normalized for creatinine level), (ii) plasma oxalate (POx) level of >5 μmol/L, and/or (iii) eGFR >45 mL/min/1.73 m².

In one aspect, the invention provides a method of treating a subject with enteric hyperoxaluria, the method comprising administering a dosing regimen of biologically active oxalate decarboxylase (OXDC) crystals to the subject (for example, up to 5 times per day), wherein, when the dosing regimen is administered to subjects having enteric hyperoxaluria and advanced chronic kidney disease (CKD), the dosing regimen causes (i) about a 25-50% mean reduction in the baseline level of 24-hour UOx excretion; and/or (b) about a 15-80% mean reduction in the baseline level of plasma oxalate (POx) level. In certain embodiments, when the dosing regimen is administered to such subjects, the dosing regimen causes (i) a reduction in kidney stone disease progression in at least about 5%, 10%, 15%, 20%, 25%, 30%, 35%, or 40% of the subjects and/or (ii) a reduction in kidney stone disease progression in a proportion of subjects that is at least about 5%, 10%, 15%, 20%, 25%, 30%, 35%, or 40% greater than the proportion of untreated subjects with a reduction in kidney stone disease progression.

In one aspect, the invention provides a method of treating a subject diagnosed as having enteric hyperoxaluria and advanced chronic kidney disease (CKD), the method comprising orally administering to the subject an effective amount of biologically active oxalate decarboxylase (OXDC) crystals up to 5 times per day; wherein the subject in need thereof has (i) UOx excretion of >40 mg/24 hours (normalized for creatinine level), (ii) plasma oxalate (POx) level of >5 μmol/L, and/or (iii) eGFR >45 mL/min/1.73 m².

In certain embodiments, administration of the OXDC crystals causes a reduction in the level of 24-hour urinary oxalate (UOx) excretion of the subject by 25-50% relative to the level of 24-hour UOx excretion prior to treatment. In certain embodiments, administration of the OXDC crystals causes a reduction in the plasma oxalate (POx) level of the subject by 15-80% relative to the level of POx prior to treatment. In certain embodiments, the OXDC crystals are administered every day for at least 12 consecutive weeks.

In certain embodiments of any of the foregoing methods, the subject has had bariatric surgery. In certain embodiments, the level of 24-hour urinary oxalate (UOx) excretion of the subject who has had bariatric surgery is reduced by at least 10%, 20%, or 30% relative to the level of 24-hour UOx excretion prior to treatment. In certain embodiments, the OXDC crystals are administered to the subject who has had bariatric surgery for at least 24 weeks, and during weeks 1-4 and 16-24 that the OXDC crystals are administered to the subject, the level of 24-hour urinary oxalate (UOx) excretion of the subject is reduced by at least 10%, 20%, or 30% relative to the level of 24-hour UOx excretion prior to treatment.

In another aspect, the invention provides a method of treating a subject with enteric hyperoxaluria who has had bariatric surgery, the method comprising administering a dosing regimen (for example, up to 5 times per day) of biologically active oxalate decarboxylase (OXDC) crystals to the subject, wherein, when the dosing regimen is administered to subjects with enteric hyperoxaluria who have had bariatric surgery, the dosing regimen causes in at least 10%, 20%, 30%, 40%, or 50% of the subjects a reduction of at least 20% in the level of 24-hour UOx excretion relative to prior to treatment. In certain embodiments, the dosing regimen is administered to the subject for at least 4 weeks, and during weeks 1-4 that the dosing regimen is administered to the subject, the dosing regimen causes in at least 10%, 20%, 30%, 40%, or 50% of the subjects a reduction of at least 20% in the level of 24-hour UOx excretion relative to prior to treatment. In certain embodiments, when the dosing regimen is administered to subjects with enteric hyperoxaluria who have had bariatric surgery, the dosing regimen causes (i) at least about a 20% mean reduction in the baseline level of 24-hour UOx excretion, and/or (ii) a reduction in kidney stone disease progression in at least about 5%, 10%, 15%, 20%, 25%, 30%, 35%, or 40% of the subjects and/or a reduction in kidney stone disease progression in a proportion of subjects that is at least about 5%, 10%, 15%, 20%, 25%, 30%, 35%, or 40% greater than the proportion of untreated subjects with a reduction in kidney stone disease progression.

In certain embodiments of any of the foregoing methods, the OXDC crystals are administered every day for at least 12, 16, 20, 24, 28, 32, 36, 40, 44, or 48 consecutive weeks, or 12, 16, 20, 24, 36, 48, 52, 54, or 60 consecutive months. For example, the OXDC crystals may be administered every day for from about 16 to about 24 consecutive weeks.

In certain embodiments of any of the foregoing methods, the subject has stage 3 CKD or stage 5 CKD. In certain embodiments, the 24-hour UOx excretion of a stage 3 CKD subject is reduced within 4 to 12 weeks after initiating treatment by 25-45%, relative to the level of 24-hour UOx excretion prior to treatment. In certain embodiments, the POx level of a stage 3 CKD subject is reduced within 4 to 12 weeks after initiating treatment by 15-45% relative to the level of POx prior to treatment. In certain embodiments, the POx level of a stage 5 CKD subject is reduced within 4 to 12 weeks after initiating treatment by 25-70% relative to the level of POx prior to treatment.

In certain embodiments of any of the foregoing methods, the OXDC crystals are administered with a food (e.g., a meal or a snack).

In certain embodiments of any of the foregoing methods, two dosage units each comprising 3,750 units of OXDC crystals are administered up to 5 times per day. In certain embodiments, about 284 mg of OXDC crystals are administered up to 5 times per day. In certain embodiments, the OXDC crystals may be spray-dried and formulated as a composition as any of a variety of physiologically acceptable salt forms, and/or with an acceptable pharmaceutical carrier and/or additive as part of a pharmaceutical composition. In certain embodiments, 120-150 mg of OXDC crystals are formulated in a capsule for oral administration.

In certain embodiments of any of the foregoing methods, the subject is a pediatric subject.

In certain embodiments of any of the foregoing methods, urine supersaturation of calcium oxalate in the subject is reduced relative to prior to treatment, for example, by at least 5%, 10%, 15%, 20%, 25%, 30%, 35%, 40%, 45%, or 50% relative to prior to treatment.

In certain embodiments of any of the foregoing methods, eGFR in the subject is reduced relative to prior to treatment, for example, by at least 15%, 20%, 25%, 30%, 35%, 40%, or 45% relative to prior to treatment.

In another aspect, the invention provides a method of treating subjects with enteric hyperoxaluria, the method comprising orally administering to the subjects an effective amount of biologically active oxalate decarboxylase (OXDC) crystals up to 5 times per day, wherein when the dosing regimen is administered to subjects, the dosing regimen causes: (a) at least about a 20% mean reduction in the baseline level of 24-hour UOx excretion; (b) about a 25-50% mean reduction in the baseline level of 24-hour UOx excretion; (c) about a 15-80% mean reduction in the baseline level of plasma oxalate (POx) level; (d) in at least 10%, 20%, 30%, 40%, or 50% of the subjects a reduction of at least 20% in the level of 24-hour UOx excretion relative to prior to treatment; and/or (e) a reduction in kidney stone disease progression in at least about 5%, 10%, 15%, 20%, 25%, 30%, 35%, or 40% of the subjects and/or a reduction in kidney stone disease progression in a proportion of subjects that is at least about 5%, 10%, 15%, 20%, 25%, 30%, 35%, or 40% greater than the proportion of untreated subjects with a reduction in kidney stone disease progression. In certain embodiments, the subjects: (a) are receiving a proton pump inhibitor and/or an acid blocker, (b) have or are at risk of developing advanced chronic kidney disease (CKD), and/or (c) have had bariatric surgery.

These and other aspects and features of the invention are described in the following detailed description and claims.

### BRIEF DESCRIPTION OF THE DRAWINGS

The invention can be more completely understood with reference to the following drawings.

FIG. 1 is a schematic depiction of the URIROX-1 study design described in Example 1.

FIG. 2 is a bar graph depicting the distribution of enteric Condition and CKD status among enrolled subjects.

FIG. 3 is a bar graph depicting the percent change in UOx for subjects receiving placebo or OXDC Crystals as indicated.

FIG. 4 is a schematic depiction of the study design described in Example 2.

FIG. 5 is a bar graph depicting the percent change in UOx and POx for the indicated subjects with CK stage 3. “Cr” in the graph represents creatinine; CKD 3Tb represents a post-kidney transplant subject with CKD 3.

FIG. 6 is a bar graph depicting the percent change in UOx and POx for the indicated subjects with CKD stage 5. Δ values presented are in μmol/L. RYGB—Roux-en-Y gastric bypass; a in the graph represents post-kidney transplant subject with CKD 5; * represents that subject had only 1 POx sample collection during the study.

### DETAILED DESCRIPTION

The invention is based, in part, upon the discovery of a dosing regimen for treating a subject with enteric hyperoxaluria or treating a subject with enteric hyperoxaluria and at risk of developing or with advanced chronic kidney disease (CKD) with an effective amount of biologically active oxalate decarboxylase (OXDC) crystals up to 5 times per day, wherein, when the dosing regimen is administered to subjects with enteric hyperoxaluria or subjects with enteric hyperoxaluria and at risk of developing or with advanced chronic kidney disease (CKD), the dosing regimen causes significant reduction in the baseline levels of 24-hour UOx excretion and plasma oxalate (POx).

Various features and aspects of the invention are discussed in more detail below.

### I. Oxalate Decarboxylase Enzymes

As used herein, oxalate decarboxylase (OXDC) (EC 4.1.1.2) refers to an oxalate carboxy-lyase enzyme. Oxalate decarboxylases are a group of enzymes known in the art to be capable of catalyzing the molecular oxygen (02) independent oxidation of oxalate to carbon dioxide and formate according to the following reaction: HO₂C—CO₂H→1CO₂+HCOOH

Isoforms of oxalate decarboxylase, and glycoforms of those isoforms, are included within this definition. OXDC from plants, bacteria and fungi are encompassed by the term, including the true oxalate decarboxylases from bacteria and fungi, such as Bacillus subtilis, Collybia velutipes or Flammulina velutipes, Aspergillus niger, Psoudomonas sp., Synechocystis sp., Streptococcus mutans, Trametes hirsute, Sclerotinia sclerotiorum, T. versicolor, Postia placenta, Myrothecium verrucaria, Agaricus bisporus, Methylobacterium extorquens, Pseudomonas oxalaticus, Ralstonia eutropha, Cupriavidus oxalaticus, Wautersia sp., Oxalicibacterium flavum, Ammoniiphilus oxalaticus, Vibrio oxalaticus, A. oxalativorans, Variovorax paradoxus, Xanthobacter autotrophicus, Aspergillus sp., Penicillium sp., and Mucor species. Optionally, the OXDC will be additionally dependent on coenzyme A, such as OXDC from organisms in the intestinal tract. In certain circumstances, OXDC is a soluble or insoluble hexameric protein.

Oxalate decarboxylases used to prepare the crystals, and which are used in methods described herein, may be isolated, for example, from a natural source, or may be derived from a natural source. As used herein, the term “derived from” means having an amino acid or nucleic acid sequence that naturally occurs in the source. For example, oxalate decarboxylase derived from Bacillus subtilis will comprise a primary sequence of a Bacillus subtilis oxalate decarboxylase protein, or will be encoded by a nucleic acid comprising a sequence found in Bacillus subtilis that encodes an oxalate decarboxylase or a degenerate thereof. A protein or nucleic acid derived from a source encompasses molecules that are isolated from the source, recombinantly produced, and/or chemically synthesized or modified. The crystals provided herein may be formed from polypeptides comprising amino acid sequences of OXDC or from a functional fragment of OXDC that retains oxalate degrading activity. Preferably, the OXDC retains at least one functional characteristic of a naturally occurring OXDC, e.g., the ability to catalyze degradation of oxalate, the ability to multimerize, and/or manganese requirement.

### Isolated Oxalate Decarboxylase

Oxalate decarboxylases have been previously isolated and are thus available from many sources, including Bacillus subtilis, Collybia velutipes or Flammulina velutipes, Aspergillus niger, Pseudomonas sp., Synechocystis sp., Streptococcus mutans, Trametes hirsute, Sclerotinia sclerotiorum, T. versicolor, Postia placenta, Myrothecium verrucaria, Agaricus bisporus, Methylobacterium extorquens, Pseudomonas oxalaticus, Ralstonia eutropha, Cupriavidus oxalaticus, Wautersia sp., Oxalicibacterium flavum, Ammoniiphilus oxalaticus, Vibrio oxalaticus, A. oxalativorans, Variovorax paradoxus, Xanthobacter autotrophicus, Aspergillus sp., Penicillium sp., and Mucor species. OXDC may also be purchased from commercial purveyors, such as, e.g., Sigma. Methods to isolate OXDC from a natural source are previously described, for example, in the following references: Tanner et al., J. Biol. Chem. 47:43627-43634 (2001); Dashek and Micales, Methods in plant biochemistry and molecular biology Boca Raton, Fla.: CRC Press 5:49-71 (1997); Magro et al., FEMS Microbiology Letters 49: 49-52 (1988); Anand et al., Biochemistry 41:7659-7669 (2002); and Tanner and Bornemann, J. Bacteriol. 182: 5271-5273 (2000). These isolated oxalate decarboxylases may be used to form the crystals and methods described herein.

### Recombinant Oxalate Decarboxylase

Alternatively, recombinant OXDCs may be used to form the crystals and methods provided herein. In some instances, recombinant OXDCs encompass or are encoded by sequences from a naturally occurring OXDC sequence. Further, OXDCs comprising an amino acid sequence that is homologous or substantially identical to a naturally occurring sequence are herein described. Also, OXDCs encoded by a nucleic acid that is homologous or substantially identical to a naturally occurring OXDC-encoding nucleic acid are provided and may be crystallized and/or administered as described herein.

Polypeptides referred to herein as “recombinant” are polypeptides which have been produced by recombinant DNA methodology, including those that are generated by procedures which rely upon a method of artificial recombination, such as the polymerase chain reaction (PCR) and/or cloning into a vector using restriction enzymes.

“Recombinant” polypeptides also include polypeptides having altered expression, such as a naturally occurring polypeptide with recombinantly modified expression in a cell, such as a host cell.

In one embodiment, OXDC is recombinantly produced from a nucleic acid that is homologous to a Bacillus subtilis or Collybia velutipes OXDC nucleic acid sequence, and sometimes it is modified, e.g., to increase or optimize recombinant production in a heterologous host. An example of such a modified sequence includes the nucleic acid sequence of the open reading frame of Collybia velutipes OXDC, for expression in Candida boldinii. The OXDC sequence may be modified to reduce its GC content, to be linked to a secretion signal sequence, e.g., an a Mating Factor secretion signal sequence, and/or to be flanked by engineered restriction endonuclease cleavage sites. In another embodiment, OXDC is recombinantly produced or from the unmodified Bacillus subtilis OXDC nucleic acid sequence which is available at GenBank Accession No:Z99120. The amino acid sequence encoded by this unmodified Bacillus subtilis OXDC nucleic acid sequence is provided as SEQ ID NO:1 as shown below.

(SEQ ID NO: 1)
MKKQNDIPQPIRGDKGATVKIPRNIERDRQNPDMLVPPETDHGTVSNMK 50
FSFSDTHNRLEKGGYAREVTVRELPISENLASVNMRLKPGAIRELHWHKE 100
AEWAYMIYGSARVTIVDEKGRSFIDDVGEGDLWYFPSGLPHSIQALEEGA 150
EFLLVFDDGSFSENSTFQLTDWLAHTPKEVIAANFGVTKEEISNLPGKEK 200
YIFENQLPGSLKDDIVEGPNGEVPYPETYRLLEQEPIESEGGKVYIADST 250
NFKVSKTIASALVTVEPGAMRELHWHPNTHEWQYYISGKARMTVFASDGH 300
ARTFNYQAGDVGYVPFAMGHYVENIGDEPLVFLEIFKDDHYADVSLNQWL 350
AMLPETFVQAHLDLGKDFTDVLSKEKHPVVKKKCSK. 385

OXDC polypeptides useful for forming OXDC crystals may be expressed in a host cell, such as a host cell comprising a nucleic acid construct that includes a coding sequence for an OXDC polypeptide or a functional fragment thereof. A suitable host cell for expression of OXDC may be yeast, bacteria, fungus, insect, plant, or mammalian cell, for example, or transgenic plants, transgenic animals or a cell-free system. In some embodiments, a host cell is capable of glycosylating the OXDC polypeptide if necessary, capable of disulfide linkages, capable of secreting the OXDC, and/or capable of supporting multimerization of OXDC polypeptides. Preferred host cells include, but are not limited to E. coli (including E. coli Origami B and E. coli BL21), Pichia pastoris, Saccharomyces cerevisiae, Schizosaccharomyces pombe, Bacillus subtilis, Aspergillus, Sf9 cells, Chinese hamster ovary (CHO), 293 cells (human embryonic kidney), and other human cells. Also transgenic plants, transgenic animals including pig, cow, goat, horse, chicken, and rabbit are suitable hosts for production of OXDC.

For recombinant production of OXDC, a host or host cell may comprise a construct in the form of a plasmid, vector, phagemid, or transcription or expression cassette that comprises at least one nucleic acid encoding an OXDC or a functional fragment thereof. A variety of constructs are available, including constructs which are maintained in single copy or multiple copy, or which become integrated into the host cell chromosome. Many recombinant expression systems, components, and reagents for recombinant expression are commercially available, for example from Invitrogen Corporation (Carlsbad, Calif.); U.S. Biological (Swampscott, Mass.); BD Biosciences Pharmingen (San Diego, Calif.); Novagen (Madison, Wis.); Stratagene (La Jolla, Calif.); and Deutsche Sammlung von Mikroorganismen and Zellkulturen GmbH (DSMZ), (Braunschweig, Germany).

Recombinant expression of OXDC is optionally controlled by a heterologous promoter, including a constitutive and/or inducible promoter. Promoters such as, e.g., T7, the alcohol oxidase (AOX) promoter, the dihydroxy-acetone synthase (DAS) promoters, the Gal 1,10 promoter, the phosphoglycerate kinase promoter, the glyceraldehyde-3-phosphate dehydrogenase promoter, alcohol dehydrogenase promoter, copper metallothionein (CUP1) promoter, acid phosphatase promoter, CMV and promoters polyhedrin are also appropriate. The particular promoter is selected based on the host or host cell. In addition, promoters that are inducible by methanol, copper sulfate, galactose, by low phosphate, by alcohol, e.g., ethanol, for example, may also be used and are well known in the art.

A nucleic acid that encodes OXDC may optionally comprise heterologous sequences. For example, a secretion sequence is included at the N-terminus of an OXDC polypeptide in some embodiments. Signal sequences such as those from a Mating Factor, BGL2, yeast acid phosphatase (PHO), xylanase, alpha amylase, from other yeast secreted proteins, and secretion signal peptides derived from other species that are capable of directing secretion from the host cell may be useful. Similarly other heterologous sequences such as linkers (e.g., comprising a cleavage or restriction endonuclease site) and one or more expression control elements, an enhancer, a terminator, a leader sequence, and one or more translation signals are within the scope of this description. These sequences may optionally be included in a construct and/or linked to the nucleic acid that encodes OXDC. Unless otherwise specified, “linked” sequences can be directly or indirectly associated with one another.

Similarly, an epitope or affinity tag such as Histidine, HA (hemagglutinin peptide), maltose binding protein, AviTag®, FLAG, or glutathione-S-transferase may be optionally linked to the OXDC polypeptide. A tag may be optionally cleavable from the OXDC after it is produced or purified. A skilled artisan can readily select appropriate heterologous sequences, for example, match host cell, construct, promoter, and/or secretion signal sequence.

OXDC homologs or variants differ from an OXDC reference sequence by one or more residues. Structurally similar amino acids can be substituted for some of the specified amino acids, for example. Structurally similar amino acids include: (I, L and V); (F and Y); (K and R); (Q and N); (D and E); and (G and A). Deletion, addition, or substitution of amino acids is also encompassed by the OXDC homologs described herein. Such homologs and variants include (i) polymorphic variants and natural or artificial mutants, (ii) modified polypeptides in which one or more residues is modified, and (iii) mutants comprising one or more modified residues.

An OXDC polypeptide or nucleic acid is “homologous” (or is a “homolog”) if it is at least 40%, 50%, 60%, 70%, 75%, 80%, 85%, 90%, 95%, 97%, 98%, 99%, or 100% identical to a reference polypeptide or nucleic acid sequence. If the homolog is not identical to the reference sequence, it is a “variant.” A homolog is “substantially identical” to a reference OXDC sequence if the nucleotide or amino acid sequence of the homolog differs from the reference sequence (e.g., by truncation, deletion, substitution, or addition) by no more than 1, 2, 3, 4, 5, 8, 10, 20, or 50 residues, and retains (or encodes a polypeptide that retains) the ability to catalyze the degradation of oxalate. Fragments of an oxalate decarboxylase may be homologs, including variants and/or substantially identical sequences. By way of example, homologs may be derived from various sources of OXDC, or they may be derived from or related to a reference sequence by truncation, deletion, substitution, or addition mutation. Percent identity between two nucleotide or amino acid sequences may be determined by standard alignment algorithms such as, for example, Basic Local Alignment Tool (BLAST) described in Altschul et al., J Mol. Biol., 215:403 410 (1990), the algorithm of Needleman et al., J. Mol. Biol., 48:444 453 (1970), or the algorithm of Meyers et al., Comput. Appl. Biosci. 4:11-17 (1988). Such algorithms are incorporated into the BLASTN, BLASTP, and “BLAST 2 Sequences” programs (reviewed in McGinnis and Madden, Nucleic Acids Res. 32:W20-W25, (2004)). When utilizing such programs, the default parameters can be used. For example, for nucleotide sequences the following settings can be used for “BLAST 2 Sequences”: program BLASTN, reward for match 2, penalty for mismatch 2, open gap and extension gap penalties 5 and 2 respectively, gap x_dropoff 50, expect 10, word size 11, filter ON. For amino acid sequences the following settings can be used for “BLAST 2 Sequences”: program BLASTP, matrix BLOSUM62, open gap and extension gap penalties 11 and 1 respectively, gap x_dropoff50, expect 10, word size 3, filter ON. The amino acid and nucleic acid sequences for OXDCs that are appropriate to form the crystals described herein may include homologous, variant, or substantially identical sequences. In some embodiments, an OXDC homolog retains at least 40%, 50%, 60%, 70%, 75%, 80%, 85%, 90%, 95%, 97%, 98%, 99%, or 100% activity relative to a reference sequence.

### Oxylate Decarboxylase Cysteine Modification

Without wishing to be bound by theory, thiol protection of C383 or elimination of the cysteine residue altogether, may enhance the formation of active oxalate decarboxylase hexamers, preventing oxidative dimerization among other oligomers. See, e.g., Tanner et al., J. Biol. Chem. 276(47):43627-34 (2001). Thiol protection or elimination of the cysteine residue of oxalate decarboxylase allows the protein to be more readily processed into crystalline form for increased potency. To reduce problems that may impact commercial scale production of oxalate decarboxylase crystals, the C383 residue may be modified by substitution of the amino acid as described in U.S. Pat. No. 8,431,122 or by deletion of C383. Alternatively, the thiol group of C383 of the oxalate decarboxylase may be modified post-translationally with a thiol protecting group to prevent it from reacting with other groups. Thiol protecting groups are well-known to those skilled in the art and are described, for example, in Greene and Wuts, Protecting Groups in Organic Synthesis, Third Edition, Wiley, N.Y., (1999), and references cited therein. For example, the thiol group of C383 may be protected by converting it to a thioether, such as, e.g., an alkyl thioether, benzyl and substituted benzyl thioether, triphenylmethyl thioether, or silyl thioether; thioester; disulfide; thiocarbonate; or thiocarbamate. Alternatively, the thiol group of the C383 residue may be protected by adding a terminal cysteine, allowing the formation of an intramolecular disulfide bridge to prevent the cysteine from reacting with other molecules. In certain embodiments, the thiol group of C383 is protected by cysteinylation. The invention provides crosslinked and/or uncrosslinked crystals of oxalate decarboxylase modified by, for example, (1) elimination of C383, (2) addition of a C-terminal cysteine, or (3) reaction with a thiol protecting group by the invention (including cysteinylation) as well as compositions comprising spray-dried OXDC crystals bearing one of these modifications.

### Purification of Oxalate Decarboxylase

Oxalate decarboxylase proteins or polypeptides may be purified from the source, such as a natural or recombinant source, prior to crystallization. A polypeptide that is referred to herein as “isolated” is a polypeptide that is substantially free of its natural environment, such as proteins, lipids, and/or nucleic acids of their source of origin (e.g., cells, tissue (i.e., plant tissue), or fluid or medium (in the case of a secreted polypeptide). Isolated polypeptides include those obtained by methods described herein or other suitable methods, and include polypeptides that are substantially pure or essentially pure, and polypeptides produced by chemical synthesis, by recombinant production, or by combinations of biological and chemical methods. Optionally, an isolated protein has undergone further processing after its production, such as by purification steps.

Purification may comprise buffer exchange and chromatographic steps. Optionally, a concentration step may be used, e.g., by dialysis, chromatofocusing chromatography, and/or associated with buffer exchange. In certain instances, cation or anion exchange chromatography is used for purification, including Q-sepharose, DEAF sepharose, DE52, sulfopropyl Sepharose chromatography or a CM52 or similar cation exchange column. Buffer exchange optionally precedes chromatographic separation, and may be performed by tangential flow filtration such as diafiltration. In certain preparations, OXDC is at least 70%, 80%, 85%, 90%, 95%, 96%, 97%, 98%, 99%, 99.5%, 99.7%, or 99.9% pure.

Purification in gram-scale runs is appropriate to prepare OXDC, and procedures are optimized for efficient, inexpensive, manufacturing-scale OXDC purification. For example, purification of at least 0.5, 1, 2, 5, 10, 20, 50, 100, 500, or 1000 grams or more of OXDC in a purification procedure is provided. In one exemplary procedure, tangential flow filtration of starting samples of at least 10 L, 50 L, 100 L, 500 L, 1000 L or more is provided, allowing buffer exchange and precipitation of contaminant proteins. A single Q-sepharose column is optionally used for purification of OXDC.

### Crystallization of Oxalate Decarboxylase

Oxalate decarboxylase crystals can be prepared using an OXDC polypeptide, such as a hexamer, as described above. See Anand et al., Biochemistry 41:7659-7669 (2002)). Vapor diffusion (such as, e.g., hanging drop and sitting drop methods), and batch methods of crystallization, for example, can be used. Oxalate decarboxylase crystals may be grown by controlled crystallization of the protein out of an aqueous solution or an aqueous solution that includes organic solvents. Conditions to be controlled include the rate of evaporation of solvent, the presence of appropriate co-solutes and buffers, pH, and temperature, for example.

For therapeutic administration, such as to treat a condition or disorder related to oxalate levels, a variety of OXDC crystal sizes are appropriate. In certain embodiments, crystals of less than about 500 μm average dimension are administered. Oxalate decarboxylase crystals with an average, maximal, or minimal dimension (for example) that is about 0.01, 0.1, 1, 5, 10, 25, 50, 100, 200, 300, 400, 500, or 1,000 μm in length are also provided.

Ranges are appropriate and would be apparent to the skilled artisan. For example, the protein crystals may have a longest dimension between about 0.01 μm and about 500 μm, alternatively, between 0.1 μm and about 50 μm. In a particular embodiment, the longest dimension ranges from about 0.1 μm to about 10 μm. Crystals may also have a shape chosen from spheres, needles, rods, plates, such as hexagons and squares, rhomboids, cubes, bipyramids and prisms. In illustrative embodiments, the crystals are cubes having a longest dimension of less than 5 μm.

In general, crystals are produced by combining the protein to be crystallized with an appropriate aqueous solvent or aqueous solvent containing appropriate crystallization agents, such as salts or organic solvents. The solvent is combined with the protein and optionally subjected to agitation at a temperature determined experimentally to be appropriate for the induction of crystallization and acceptable for the maintenance of protein activity and stability. The solvent can optionally include co-solutes, such as monovalent or divalent cations, co-factors or chaotropes, as well as buffer species to control pH. The need for co-solutes and their concentrations are determined experimentally to facilitate crystallization. In an industrial scale process, the controlled precipitation leading to crystallization can be carried out by the combination of protein, precipitant, co-solutes and, optionally, buffers in a batch process, for example. Alternative laboratory crystallization methods and conditions, such as dialysis or vapor diffusion, can be adopted (McPherson, et al., Methods Enzymol. 114:112-20 (1985) and Gilliland, Crystal Growth 90:51-59 (1998)). Occasionally, incompatibility between the cross-linking agent and the crystallization medium might require changing the buffers (solvent) prior to cross-linking.

As set forth in the Examples, oxalate decarboxylase crystallizes under a number of conditions, including a wide pH range (e.g., pH 3.5 to 8.0). A precipitant such as a polyethylene glycol (such as, e.g., PEG 200, PEG 400, PEG 600, PEG 1000, PEG 2000, PEG 3000, PEG 8000) or an organic cosolvent such as 2-methyl-2,4-pentanedial (MPD) is included in some embodiments as described. Common salts that may be used include sodium chloride, potassium chloride, ammonia sulfate, zinc acetate, etc. Oxalate decarboxylase may be at a concentration of, e.g., at least 5, 10, 15, 20, 25, 30, 35, 40, 45, 50, 60, 70, 80, 90, or 100 mg/ml, or more in a crystallization broth. The efficiency or yield of a crystallization reaction is at least 50%, 60%, 70%, 80%, 90%, 91%, 92%, 93%, 94%, 95%, 96%, 97% 98%, 99%, or more. In one embodiment, crystals of oxalate decarboxylase are grown or produced by a batch process by mixing a solution of oxalate decarboxylase with an appropriate buffer.

Crystallization from Cells or Cell Extract

Crystals may be prepared directly from cells or crude cell extracts. In one embodiment, bacteria cells expressing oxalate decarboxylase are harvested. Cells are resuspended with or without DNase and homogenized. A salt solution is added to the cell lysis to reach a salt concentration of about 0.3 M, 0.4 M, 0.5 M, 0.6 M, up to 2.5 M, 3.0 M, 3.5 M, 4.0 M, or more. The salt added can be a sodium salt, a potassium salt, a calcium salt, or other salts. Proteins may be optionally extracted from the cell mixture by removing cell debris. In one embodiment, homogenized cell mixture is centrifuged, leaving proteins in the supernatant solution. Crystals are generated by reducing salt concentration of the cell mixture or protein solution. In one embodiment, salt is removed through dialysis to maintain protein concentration. To increase crystal yield, protein solution may be concentrated before salt concentration of the solution is reduced. Crystals may be generated at a solution with a pH of about 6, 7, 8 or 9.

Crystals may be prepared from a protein precipitate or pellet. In one embodiment, cells expressing desired proteins are harvested and oxalate decarboxylase protein is collected in a precipitate or pellet. Pellet or precipitate containing oxalate decarboxylase protein is solubilized in a salt solution. Crystals are formed by reducing salt concentration in the protein solution. For increased crystal yields, the salt concentration in the solubilized protein solution is at least about 0.3 M, 0.4 M, 0.5 M or more before it is reduced to produce crystals.

Crystals may also be prepared from a protein solution. In one embodiment, an oxalate decarboxylase protein solution is concentrated in a salt solution, and crystals are formed when the salt concentration in the solution is reduced. For increased crystal yields, the salt concentration is at least about 0.3 M, 0.4 M, 0.5 M or more before it is reduced to produce crystals.

### Compositions Comprising OXDC Crystals

In certain embodiments, the OXDC crystals are provided as a composition, such as a pharmaceutical composition (see, e.g., U.S. Pat. No. 6,541,606, describing formulations and compositions of protein crystals). In certain embodiments, the pharmaceutical compositions comprising spray-dried OXDC crystals may include one or more ingredients or excipients, including, but not limited to sugars and biocompatible polymers. Examples of excipients are described in Handbook of Pharmaceutical Excipients, published jointly by the American Pharmaceutical Association and the Pharmaceutical Society of Great Britain, and further examples are set forth below.

In certain embodiments, the OXDC crystals have a Hausner ratio of between 1.00 to 1.59 (e.g., 1-1.59, 1-1.55, 1-1.50, 1-1.45, 1-1.40, 1-1.35, 1-1.30, 1-1.25, 1-1.20, 1-1.15, 1-1.10, 1.10-1.59, 1.15-1.59, 1.20-1.59, 1.25-1.59, 1.30-1.59, 1.35-1.59, 1.40-1.59, 1.45-1.59, 1.50-1.59, or 1.55-1.59), or between 1.12 to 1.40 (e.g., 1.12-1.40, 1.12-1.35, 1.12-1.30, 1.12-1.25, or 1.12-1.20). In certain embodiments, the OXDC crystals have a Hausner ratio of approximately 1.34.

The OXDC enzyme may be administered as a spray-dried crystal in a composition as any of a variety of physiologically acceptable salt forms, and/or with an acceptable pharmaceutical carrier and/or additive as part of a pharmaceutical composition.

Physiologically acceptable salt forms and standard pharmaceutical formulation techniques and excipients are well known to persons skilled in the art (see, e.g., Physician's Desk Reference (PDR) 2003, 57th ed., Medical Economics Company, 2002; and Remington: The Science and Practice of Pharmacy, eds. Gennado et al., 20th ed, Lippincott, Williams & Wilkins, 2000). For the purposes of this application, “formulations” include “crystal formulations.”

Oxalate decarboxylase useful in the methods of the present disclosure may be combined with an excipient. According to the present disclosure, an “excipient” acts as a filler or a combination of fillers used in pharmaceutical compositions. Exemplary ingredients and excipients for use in the compositions are set forth as follows.

### Sugars

The sugar used as an excipient may be a monosaccharide, disaccharide, oligosaccharide, or polysaccharide. Exemplary monosaccharides include but are not limited to ribose, arabinose, xylose, lyxose, allose, altrose, glucose, mannose, fructose, iodose, galactose, xylitol, sucralose and talose. Exemplary disaccharides include but are not limited to sucrose, lactose, maltose, lactulose, trehalose, and cellobiose.

### Biodegradable Polymers

Biodegradable polymers degrade by hydrolysis or solubilization may be included in OXDC crystal compositions. Degradation can be heterogenous (occurring primarily at the particle surface), or homogenous (degrading evenly throughout the polymer matrix). Ingredients such as one or more excipients or pharmaceutical ingredients or excipients may be included in OXDC crystal compositions. An ingredient may be an inert or active ingredient.

In some embodiments, the pharmaceutical composition comprises crystallized oxalate decarboxylase which is spray-dried. In other embodiments, an excipient is added to the crystallized oxalate decarboxylase, and then the mixture is spray-dried to form the pharmaceutical composition. In certain embodiments, the excipient is a sugar. In certain embodiments, the sugar is a monosaccharide or a disaccharide. In some embodiments the excipient is trehalose, sucrose, or glucose. In certain embodiments the excipient is trehalose.

In an exemplary embodiment, trehalose is added to oxalate decarboxylase crystals and the mixture is spray-dried to form a pharmaceutical composition.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases, see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

It is contemplated that a disclosed oxalate decarboxylase may be modified, engineered or chemically conjugated. For example, it is contemplated that a disclosed oxalate decarboxylase can be conjugated to an effector agent using standard in vitro conjugation chemistries. If the effector agent is a polypeptide, the oxalate decarboxylase enzyme can be chemically conjugated to the effector or joined to the effector as a fusion protein. Construction of fusion proteins is within ordinary skill in the art.

### II. Pharmaceutical Compositions

For therapeutic use, an oxalate decarboxylase enzyme (e.g., oxalate decarboxylase crystals) described herein preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing an oxalate decarboxylase enzyme (e.g., oxalate decarboxylase crystals) disclosed herein can be presented in a dosage unit form and can be prepared by any suitable method. A pharmaceutical composition should be formulated to be compatible with its intended route of administration. The pharmaceutical compositions may be in a variety of forms. These include, for example, liquid, semi-solid and solid dosage forms, such as liquid solutions, dispersions or suspensions, tablets, pills, powders, liposomes and suppositories. The preferred form will depend upon the intended mode of administration and therapeutic application.

Although the compositions preferably are formulated for administration enterally (for example, orally), such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and infrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Depending upon the mode of administration, for example, by parenteral administration, it may be desirable to produce a pharmaceutical formulation that is sterile. Sterilization can be accomplished by any suitable method, e.g., filtration through sterile filtration membranes. Where the composition is lyophilized, filter sterilization can be conducted prior to or following lyophilization and reconstitution.

### III. Therapeutic Uses

The methods and compositions disclosed herein can be used to treat various diseases or disorders associated with an elevated amount of oxalate in a subject. As used herein, “elevated amount of oxalate in a subject” may refer to an elevated amount of oxalate in a body fluid (e.g., blood, plasma, serum, or urine), tissue and/or cell in a subject, relative to a subject without the disease or disorder.

The term “effective amount” as used herein refers to the amount of an active agent (e.g., oxalate decarboxylase crystals) sufficient to effect beneficial or desired results. An effective amount can be administered in one or more administrations, applications or dosages and is not intended to be limited to a particular formulation or administration route.

“Dosing regimen” as used herein means delivery of an effective amount of an active agent (e.g., oxalate decarboxylase crystals) over a period of time so as to treat the desired indication. Exemplary dosing regimens can be found in Examples 1 and 2 below.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of diseases or disorders associated with an elevated amount of oxalate include primary hyperoxaluria, enteric hyperoxaluria, idiopathic hyperoxaluria, ethylene glycol poisoning, cystic fibrosis, inflammatory bowel disease, urolithiasis, nephrolithiasis, chronic kidney disease, hemodialysis, gastrointestinal bypass, and kidney stones.

In certain embodiments, a method disclosed herein can be used to treat a subject with enteric hyperoxaluria and/or with advanced chronic kidney disease or at risk of developing advanced chronic kidney disease (CKD). For example, a subject may have stage 3 or stage 5 CKD.

In certain embodiments, a method disclosed herein can be used to treat a subject who is receiving a proton pump inhibitor or has received a proton pump inhibitor. Exemplary proton pump inhibitors include pantoprazole (Protonix), lansoprazole (Prevacid), esomeprazole (Nexium), omeprazole (Prilosec), rabeprazole, prostaglandins (such as misoprostoL (Cytotec)), sucralfate, and antacids.

In certain embodiments, a method disclosed herein can be used to treat a subject who is receiving an acid blocker or has received an acid blocker. Exemplary acid blockers include H2 blockers (such as cimetidine (Tagamet), ranitidine (Zantac), famotidine (Pepcid), and nizatidine (Axid)).

In certain embodiments, a method disclosed herein can be used to treat a subject who has had bariatric surgery.

In certain embodiments, a method disclosed herein can be used to treat a subject who has or has had a urinary oxalate (UOx) excretion level of greater than or equal to 30 mg/24 hours, 40 mg/24 hours, 50 mg/24 hours, or 60 mg/24 hours (normalized for creatinine level).

In certain embodiments, a method disclosed herein can be used to treat a subject who has or has had a plasma oxalate (POx) level of greater than or equal to 3 μmol/L, 4 μmol/L, 5 μmol/L, 6 μmol/L, 7 μmol/L, 8 μmol/L, 9 μmol/L, or 10 μmol/L.

In certain embodiments, a method disclosed herein can be used to treat a subject who has or has had an estimated glomerular filtration rate (eGFR) less than or equal to 30 mL/min/1.73 m², 35 mL/min/1.73 m², 40 mL/min/1.73 m², 45 mL/min/1.73 m², 50 mL/min/1.73 m², 55 mL/min/1.73 m², or 60 mL/min/1.73 m².

In certain embodiments, a disclosed method or dosing regimen comprises administering OXDC crystals 1, 2, 3, 4, 5, 6, 7, 8, 9, or 10 times per day. In certain embodiments, a disclosed method or dosing regimen comprises administering OXDC crystals every day for at least 14, 28, 42, 56, or 70 days, at least 12, 16, 20, 24, 28, 32, 36, 40, 44, or 48 consecutive weeks, or at least 12, 16, 20, 24, 36, 48, 52, 54, or 60 consecutive months. For example, the OXDC crystals may be administered every day for from about 12 to about 48 weeks, about 12 to about 40 weeks, about 12 to about 32 weeks, about 12 to about 24 weeks, about 12 to about 16 weeks, about 16 to about 48 weeks, about 16 to about 40 weeks, about 16 to about 32 weeks, about 16 to about 24 weeks, about 24 to about 48 weeks, about 24 to about 40 weeks, about 24 to about 32 weeks, about 32 to about 48 weeks, about 32 to about 40 weeks, about 12 to about 60 months, about 12 to about 48 months, about 12 to about 36 months, about 12 to about 24 months, about 24 to about 60 months, about 24 to about 48 months, about 24 to about 36 months, about 36 to about 60 months, about 36 to about 48 months, or about 48 to about 60 months.

In certain embodiments, a disclosed method or dosing regimen comprises administering to the subject two dosage units each comprising from about 3,600 units to about 3,900 units, from about 3,600 units to about 3,800 units, from about 3,600 units to about 3,700 units, from about 3,700 units to about 3,900 units, from about 3,700 units to about 3,800 units, from about 3,800 to about 3,900 units, about 3,600 units, about 3,650 units, about 3,700 units, about 3,750 units, about 3,800 units, about 3,850 units, or about 3,900 units of OXDC crystals, for example, 1, 2, 3, 4, 5, 6, 7, 8, 9, 10 times per day.

In certain embodiments, a disclosed method or dosing regimen comprises administering to the subject two dosage units comprising from about 272 mg to about 296 mg, from about 272 mg to about 292 mg, from about 272 mg to about 288 mg, from about 272 mg to about 284 mg, from about 272 mg to about 280 mg, from about 272 mg to about 276 mg, from about 276 mg to about 296 mg, from about 276 mg to about 292 mg, from about 276 mg to about 288 mg, from about 276 mg to about 284 mg, from about 276 mg to about 280 mg, from about 280 mg to about 296 mg, from about 280 mg to about 292 mg, from about 280 mg to about 288 mg, from about 280 mg to about 284 mg, from about 284 mg to about 296 mg, from about 284 mg to about 292 mg, from about 284 mg to about 288 mg, from about 288 mg to about 296 mg, from about 288 mg to about 292 mg, from about 292 mg to about 296 mg, about 272 mg, about 276 mg, about 280 mg, about 284 mg, about 288 mg, about 292 mg, or about 296 mg of OXDC crystals, for example, 1, 2, 3, 4, 5, 6, 7, 8, 9, 10 times per day.

In certain embodiments, a disclosed method or dosing regimen comprises administering to the subject from about 110 mg to about 160 mg, from about 110 mg to about 150 mg, from about 110 mg to about 140 mg, from about 110 mg to about 130 mg, from about 110 mg to about 120 mg, from about 120 mg to about 160 mg, from about 120 mg to about 150 mg, from about 120 mg to about 140 mg, from about 120 mg to about 130 mg, from about 130 mg to about 160 mg, from about 130 mg to about 150 mg, from about 130 mg to about 140 mg, from about 140 mg to about 160 mg, from about 140 mg to about 150 mg, from about 150 mg to about 160 mg, about 110 mg, about 120 mg, about 130 mg, about 140 mg, about 150 mg, or about 160 mg of OXDC crystals formulated in a capsule for oral administration.

In certain embodiments, a disclosed method or dosing regimen causes a reduction (e.g., a significant reduction) in a level of 24-hour urinary oxalate (UOx) excretion. For example, the disclosed method or dosing regimen may reduce 24-hour UOx excretion in a subject by from about 10% to about 60%, about 10% to about 50%, about 10% to about 40%, about 10% to about 30%, about 10% to about 25%, about 10% to about 20%, about 10% to about 15%, about 15% to about 60%, about 15% to about 50%, about 15% to about 40%, about 15% to about 30%, about 15% to about 25%, about 15% to about 20%, about 20% to about 60%, about 20% to about 50%, about 20% to about 40%, about 20% to about 30%, about 20% to about 25%, about 25% to about 60%, about 25% to about 50%, about 25% to about 40%, about 25% to about 30%, about 30% to about 60%, about 30% to about 50%, about 30% to about 40%, about 40% to about 60%, about 40% to about 50%, about 50% to about 60%, at least 10%, at least 15%, at least 20%, at least 25%, at least 30%, at least 40%, at least 50%, or least 60% relative to the level of 24-hour UOx excretion prior to treatment.

In certain embodiments, when a disclosed method or dosing regimen is administered to subjects, the method or dosing regimen causes a reduction (e.g., a significant reduction) in a mean level of 24-hour urinary oxalate (UOx) excretion. For example, the disclosed method or dosing regimen may reduce mean 24-hour UOx excretion in the subjects by from about 10% to about 60%, about 10% to about 50%, about 10% to about 40%, about 10% to about 30%, about 10% to about 25%, about 10% to about 20%, about 10% to about 15%, about 15% to about 60%, about 15% to about 50%, about 15% to about 40%, about 15% to about 30%, about 15% to about 25%, about 15% to about 20%, about 20% to about 60%, about 20% to about 50%, about 20% to about 40%, about 20% to about 30%, about 20% to about 25%, about 25% to about 60%, about 25% to about 50%, about 25% to about 40%, about 25% to about 30%, about 30% to about 60%, about 30% to about 50%, about 30% to about 40%, about 40% to about 60%, about 40% to about 50%, about 50% to about 60%, at least 10%, at least 15%, at least 20%, at least 25%, at least 30%, at least 40%, at least 50%, or least 60% relative to the mean 24-hour UOx excretion in the subjects prior to treatment.

In certain embodiments, a disclosed method or dosing regimen causes a reduction (e.g., a significant reduction) in plasma oxalate (POx). For example, the disclosed method or dosing regimen may reduce POx in a subject by from about 10% to about 90%, about 10% to about 80%, about 10% to about 70%, about 10% to about 60%, about 10% to about 50%, about 10% to about 40%, about 10% to about 30%, about 10% to about 25%, about 10% to about 20%, about 10% to about 15%, about 15% to about 90%, about 15% to about 80%, about 15% to about 70%, about 15% to about 60%, about 15% to about 50%, about 15% to about 40%, about 15% to about 30%, about 15% to about 25%, about 15% to about 20%, about 20% to about 90%, about 20% to about 80%, about 20% to about 70%, about 20% to about 60%, about 20% to about 50%, about 20% to about 40%, about 20% to about 30%, about 20% to about 25%, about 25% to about 90%, about 25% to about 80%, about 25% to about 70%, about 25% to about 60%, about 25% to about 50%, about 25% to about 40%, about 25% to about 30%, about 30% to about 90%, about 30% to about 80%, about 30% to about 70%, about 30% to about 60%, about 30% to about 50%, about 30% to about 40%, about 40% to about 90%, about 40% to about 80%, about 40% to about 70%, about 40% to about 60%, about 40% to about 50%, about 50% to about 90%, about 50% to about 80%, about 50% to about 70%, about 50% to about 60%, about 60% to about 90%, about 60% to about 80%, about 60% to about 70%, about 70% to about 90%, about 70% to about 80%, about 80% to about 90%, at least 10%, at least 15%, at least 20%, at least 25%, at least 30%, at least 40%, at least 50%, least 60%, at least 70%, at least 80%, or least 90% relative to the level of POx prior to treatment.

In certain embodiments, when a disclosed method or dosing regimen is administered to subjects, the method or dosing regimen causes a reduction (e.g., a significant reduction) in a mean level of POx. For example, the disclosed method or dosing regimen may reduce the mean POx in the subjects by from about 10% to about 90%, about 10% to about 80%, about 10% to about 70%, about 10% to about 60%, about 10% to about 50%, about 10% to about 40%, about 10% to about 30%, about 10% to about 25%, about 10% to about 20%, about 10% to about 15%, about 15% to about 90%, about 15% to about 80%, about 15% to about 70%, about 15% to about 60%, about 15% to about 50%, about 15% to about 40%, about 15% to about 30%, about 15% to about 25%, about 15% to about 20%, about 20% to about 90%, about 20% to about 80%, about 20% to about 70%, about 20% to about 60%, about 20% to about 50%, about 20% to about 40%, about 20% to about 30%, about 20% to about 25%, about 25% to about 90%, about 25% to about 80%, about 25% to about 70%, about 25% to about 60%, about 25% to about 50%, about 25% to about 40%, about 25% to about 30%, about 30% to about 90%, about 30% to about 80%, about 30% to about 70%, about 30% to about 60%, about 30% to about 50%, about 30% to about 40%, about 40% to about 90%, about 40% to about 80%, about 40% to about 70%, about 40% to about 60%, about 40% to about 50%, about 50% to about 90%, about 50% to about 80%, about 50% to about 70%, about 50% to about 60%, about 60% to about 90%, about 60% to about 80%, about 60% to about 70%, about 70% to about 90%, about 70% to about 80%, about 80% to about 90%, at least 10%, at least 15%, at least 20%, at least 25%, at least 30%, at least 40%, at least 50%, least 60%, at least 70%, at least 80%, or least 90% relative to the mean POx in the subjects prior to treatment.

In certain embodiments, a disclosed method or dosing regimen causes a reduction in 24-hour urinary oxalate (UOx) excretion and/or plasma oxalate (POx) in a subject or subjects within about 1, 2, 3, 4, 5, 6, or 7 days, or 1, 2, 3 or 4 weeks of an administration (e.g., the initial administration) of the method or dosing regimen.

In certain embodiments, when a disclosed method or dosing regimen is administered to subjects, the method or dosing regimen causes a reduction (e.g., a significant reduction) in kidney stone disease progression. For example, the disclosed method or dosing regimen may reduce kidney stone disease progression in from about 10% to about 60%, about 10% to about 50%, about 10% to about 40%, about 10% to about 30%, about 10% to about 25%, about 10% to about 20%, about 10% to about 15%, about 15% to about 60%, about 15% to about 50%, about 15% to about 40%, about 15% to about 30%, about 15% to about 25%, about 15% to about 20%, about 20% to about 60%, about 20% to about 50%, about 20% to about 40%, about 20% to about 30%, about 20% to about 25%, about 25% to about 60%, about 25% to about 50%, about 25% to about 40%, about 25% to about 30%, about 30% to about 60%, about 30% to about 50%, about 30% to about 40%, about 40% to about 60%, about 40% to about 50%, about 50% to about 60%, at least 5%, at least 10%, at least 15%, at least 20%, at least 25%, at least 30%, at least 35%, at least 40%, at least 50%, or least 60% of the subjects. Alternatively, or in addition, the disclosed method or dosing regimen may, for example, cause a reduction in kidney stone disease progression in a proportion of subjects that is at least about 10% to about 60%, about 10% to about 50%, about 10% to about 40%, about 10% to about 30%, about 10% to about 25%, about 10% to about 20%, about 10% to about 15%, about 15% to about 60%, about 15% to about 50%, about 15% to about 40%, about 15% to about 30%, about 15% to about 25%, about 15% to about 20%, about 20% to about 60%, about 20% to about 50%, about 20% to about 40%, about 20% to about 30%, about 20% to about 25%, about 25% to about 60%, about 25% to about 50%, about 25% to about 40%, about 25% to about 30%, about 30% to about 60%, about 30% to about 50%, about 30% to about 40%, about 40% to about 60%, about 40% to about 50%, about 50% to about 60%, at least 5%, at least 10%, at least 15%, at least 20%, at least 25%, at least 30%, at least 35%, at least 40%, at least 50%, or least 60% greater than the proportion of untreated subjects with a reduction in kidney stone disease progression.

As used herein “kidney stone disease progression” refers to any increase in the size or number of kidney stones in a subject or any increase in symptoms associated with or mediated by kidney stones.

Kidney stone disease progression may be measured by any method known in the art. One or more of the following factors may be considered in assaying kidney stone disease progression: (i) renal colic (i.e., abdominal and/or flank pain), (ii) hematuria, (iii) a passed stone reported by a subject, (iv) a new kidney or ureteral stone as shown by imaging, (v) a new sign of obstruction (i.e., unilateral collecting system or ureteral dilatation, consistent with recent stone passage) as shown by imaging, (vi) in a subject with a kidney stone at baseline (or the previous imaging time point), an enlarged stone (e.g., an increase in the size of the kidney stones compared with baseline or the previous imaging time point, as relevant) as shown by imaging, and (vii) any previously unplanned intervention for removal of a new stone.

Imaging tests to measure kidney stone disease progression include renal ultrasound (RUS), kidney-ureter-bladder X-ray (KUB), and low dose computed tomography (CT). The use of these methods to identify and quantify kidney stones is well known, and is described, for example, in Mitterberger et al. (2007) BJU INT. 100(4):887-890, Ripolles et al. (2004) EUR. RADIOL. 14(1):129-136, Assimos et al. (2016) J. UROL. 196(4):1153-1160, Fulgham et al. (2013) J UROL. 189(4):1203-1213, and Turk et al. (2015) EUR. UROL. 69(3):468-474.

In certain embodiments, for a subject with a symptomatic kidney stone, kidney stone disease progression is defined as the combination of: (a) renal colic (i.e., abdominal and/or flank pain) or hematuria; and (b) at least one of (i) a passed stone reported by the subject, and in a subject with a kidney stone at baseline (or the previous imaging time point), it is confirmed to be a new stone by imaging; (ii) imaging shows a new kidney or ureteral stone, or a new sign of obstruction (i.e., unilateral collecting system or ureteral dilatation, consistent with recent stone passage), and in a subject with a kidney stone at baseline (or the previous imaging time point), a new or enlarged stone (e.g., an increase in the size or number of kidney stones seen on imaging compared with baseline or the previous imaging time point, as relevant); or (iii) any previously unplanned intervention for removal of a new stone, and in a subject with a kidney stone at baseline (or the previous imaging time point), a new or enlarged stone. In certain embodiments, for a subject with an asymptomatic kidney stone, kidney stone disease progression is defined as a new stone (previously undetected) or an enlarged stone, as revealed by imaging.

Additional methods to assay kidney stone disease progression based on a composite of a symptomatic stone event or the presence of new or enlarged kidney stone(s) detected by imaging are described in Borghi et al. (2002) N. ENGL. J. MED. 346(2):77-84, Dussol et al. (2008) NEPHRON. CLIN. PRACT. 110(3):c185-194, and Ettinger et al. (1997) THE JOURNAL OF UROLOGY 158(6):2069-2073.

In certain embodiments, a disclosed method or dosing regimen causes a reduction (e.g., a significant reduction) in urine supersaturation of calcium oxalate in a subject. For example, urine supersaturation of calcium oxalate in the subject may be reduced by from about 10% to about 60%, about 10% to about 50%, about 10% to about 40%, about 10% to about 30%, about 10% to about 25%, about 10% to about 20%, about 10% to about 15%, about 15% to about 60%, about 15% to about 50%, about 15% to about 40%, about 15% to about 30%, about 15% to about 25%, about 15% to about 20%, about 20% to about 60%, about 20% to about 50%, about 20% to about 40%, about 20% to about 30%, about 20% to about 25%, about 25% to about 60%, about 25% to about 50%, about 25% to about 40%, about 25% to about 30%, about 30% to about 60%, about 30% to about 50%, about 30% to about 40%, about 40% to about 60%, about 40% to about 50%, about 50% to about 60%, at least 5%, at least 10%, at least 15%, at least 20%, at least 25%, at least 30%, at least 35%, at least 40%, at least 50%, or least 60% relative to prior to treatment.

In certain embodiments, a disclosed method or dosing regimen causes a reduction (e.g., a significant reduction) in estimated glomerular filtration rate (eGFR) in a subject. For example, eGFR in the subject may be reduced by about 10% to about 60%, about 10% to about 50%, about 10% to about 40%, about 10% to about 30%, about 10% to about 25%, about 10% to about 20%, about 10% to about 15%, about 15% to about 60%, about 15% to about 50%, about 15% to about 40%, about 15% to about 30%, about 15% to about 25%, about 15% to about 20%, about 20% to about 60%, about 20% to about 50%, about 20% to about 40%, about 20% to about 30%, about 20% to about 25%, about 25% to about 60%, about 25% to about 50%, about 25% to about 40%, about 25% to about 30%, about 30% to about 60%, about 30% to about 50%, about 30% to about 40%, about 40% to about 60%, about 40% to about 50%, about 50% to about 60%, at least 5%, at least 10%, at least 15%, at least 20%, at least 25%, at least 30%, at least 35%, at least 40%, at least 50%, or least 60% relative to prior to treatment.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the subject overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This example describes a clinical study to determine the efficacy of OXDC Crystals in reducing UOx excretion in subjects with enteric HOx and evaluate the safety of OXDC Crystals in subjects with enteric HOx.

### Background

Oxalate is an end-product of carbohydrate and amino acid metabolism, and it is also absorbed from the diet. There is no known physiological requirement for oxalate, and the metabolic and dietary oxalate load is excreted unchanged in the urine. Hyperoxaluria (HOx) is a serious metabolic disorder and one of the major risk factors for progression of kidney stone disease and can also lead to chronic kidney disease and end stage kidney disease. Enteric HOx refers to excessive urine oxalate (UOx) excretion that is a complication of increased intestinal oxalate absorption due to an underlying gastrointestinal (GI) condition associated with malabsorption (e.g., bariatric surgery, short bowel syndrome, inflammatory bowel disease, etc.) with UOx levels often approaching levels in primary hyperoxaluria. The clinical literature suggests that a 20% reduction in 24-hour UOx would translate to a 25%-50% reduction in the risk of kidney stone (KS) disease.

There are no approved pharmacological therapies for HOx. Current management includes recommendations to restrict dietary oxalate and increase calcium and fluid intake, but these may be difficult to sustain or may be of limited efficacy.

OXDC Crystals (ALLN-177) are an oral enzyme therapy that specifically targets oxalate: OXDC Crystals degrade oxalate within the GI tract, resulting in less oxalate available for systemic absorption, thereby reducing UOx excretion. OXDC Crystals are not systemically absorbed to any meaningful extent

### Study Design

URIROX-1 (NCT03456830) was a global study conducted at >30 participating sites in Canada, France, Germany, Italy, Spain, United Kingdom, and United States. It was a Phase 3, multi-center, randomized, double-blind, placebo-controlled study. The planned enrollment was 124 subjects (to allow for 10% drop-out). Subjects were randomized 1:1 to either OXDC Crystals or placebo. The OXDC Crystals administered are spray-dried crystals of recombinant OXDC from Bacillus subtilis having an amino acid sequence of SEQ ID NO:1, in which Cys383 is protected with a thiol protecting group. The dose regimen was 2 capsules of OXDC Crystals (3,750 units/capsule) 3-5 times per day with each meal/snack for 4 weeks, or matching placebo. Two 24-hour urine collections were performed each week during treatment.

Randomization was stratified by: bariatric surgery vs. other enteric condition; baseline UOx <90 vs ≥90 mg/24 h; or use of proton pump inhibitors (PPI)/acid (H2) blockers vs. not.

The key inclusion criteria were: 18 years of age or older; history of HOx, secondary to a known underlying enteric disorder associated with malabsorption (e.g., bariatric surgery, Crohn's disease, short bowel syndrome, or other malabsorption syndrome); has adequate 24-hour urine collection at Screening, with resulting UOx≥50 mg/24 hr; two adequate 24-hour urine collections at Baseline, with average UOx≥50 mg/24 hr (and neither is <40 mg/24 hr); and if taking concomitant medications for management of kidney stone risk factors, dose regimen must be stable for ≥8 weeks.

The key exclusion criteria were: has >30% variability in the ratio of creatinine (mg)/body weight (kg) among the three 24-hour urine samples collected prior to randomization (1 at Screening, 2 at Baseline); unable or unwilling to discontinue Vitamin C supplementation; is in acute renal failure or has an estimated glomerular filtration rate (eGFR)<30 mL/minute/1.73 m2 at Screening; has an active autoimmune disorder or other condition requiring therapy with high doses of systemic steroids (i.e., >10 mg/day prednisone or equivalent) or intensification of other immunosuppressant therapy within 4 weeks prior to or during Screening; has received study drug (OXDC Crystals or placebo) in any other ALLN-177 clinical study, or participation in another drug or device clinical trial within 30 days prior to or during Screening.

The primary endpoint was percent change from Baseline in 24-hour UOx excretion during Weeks 1-4 (average). The secondary endpoints were: proportion of subjects with a ≥20% reduction from Baseline in 24-hour UOx excretion during Weeks 1-4; and analysis of efficacy parameters by bariatric surgery vs. other enteric condition subgroup. The exploratory endpoint was change from Baseline to Week 4 in urine supersaturation of calcium oxalate. The safety endpoints were: treatment-emergent adverse events (TEAEs); AEs of special interest (passage of kidney stones, procedures, hospitalizations, or emergency room visits related to kidney stones); and subgroup analyses of TEAEs and other selected safety parameters by underlying condition.

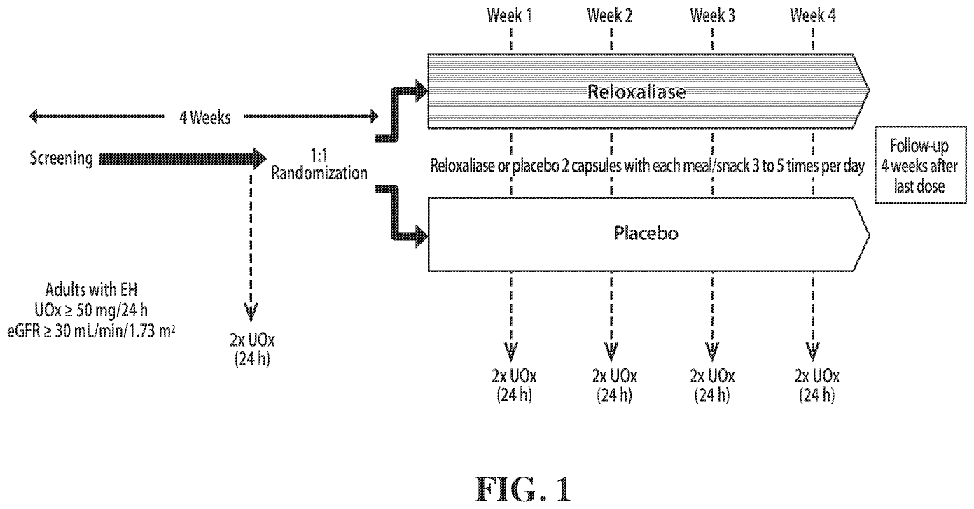

The general study design is depicted in FIG. 1.

### Results

Of 222 subjects screened, 115 were randomized and 114 completed the study. 1 subject from the placebo group discontinued prematurely. Subjects' mean age was 59 years and 48% were female. Demographic and baseline characteristics are summarized in TABLE 1.

TABLE 1
Baseline Demographics and Characteristics

| | OXDC | | | |
|---|---|---|---|---|
| | Crystals | | Placebo | |
| Category/Statistic | (N = 58) | | (N = 57) | |
| Age (years), mean (SD) | 58.7 | (10.09) | 58.6 | (10.18) |
| Gender, n (%) Female | 28 | (48.3) | 27 | (47.4) |
| Race - Caucasian, n (%) | 56 | (96.6) | 54 | (94.7) |
| Race - Black or African | 0 | | 3 | (5.3) |
| American, n (%) | | | | |
| Race - American Indian or | 1 | (1.7) | 0 | |
| Alaska Native, n (%) | | | | |
| Race - Other, n (%) | 1 | (1.7) | 0 | |
| Enteric condition - Bariatric | 40 | (69.0) | 38 | (66.7) |
| surgery, n (%) [Roux-en-Y | [27 | (46.6)] | [27 | (47.4)] |
| gastric bypass, n (%)] | | | | |
| Enteric condition - IBD, n (%) | 10 | (17.2) | 10 | (17.5) |
| Enteric condition - SBS, n (%) | 3 | (5.2) | 8 | (14.0) |
| Enteric condition - Pancreatic | 3 | (5.2) | 0 | |
| insufficiency, n (%) | | | | |
| Enteric condition - Other, n (%) | 2 | (3.4) | 1 | (1.8) |
| Baseline UOx (mg/24 h), | 87.3 | (28.87) | 91.1 | (41.64) |
| mean (SD) | | | | |
| Baseline UOx ≥ 90 mg/24 h, | 22 | (37.9) | 23 | (40.4) |
| n (%) | | | | |
| eGFR (mL/min/1.73 m2), | 76.4 | (22.71) | 80.5 | (24.60) |
| mean (SD) | | | | |
| CKD stage 3, n (%) | 16 | (27.6) | 14 | (24.6) |

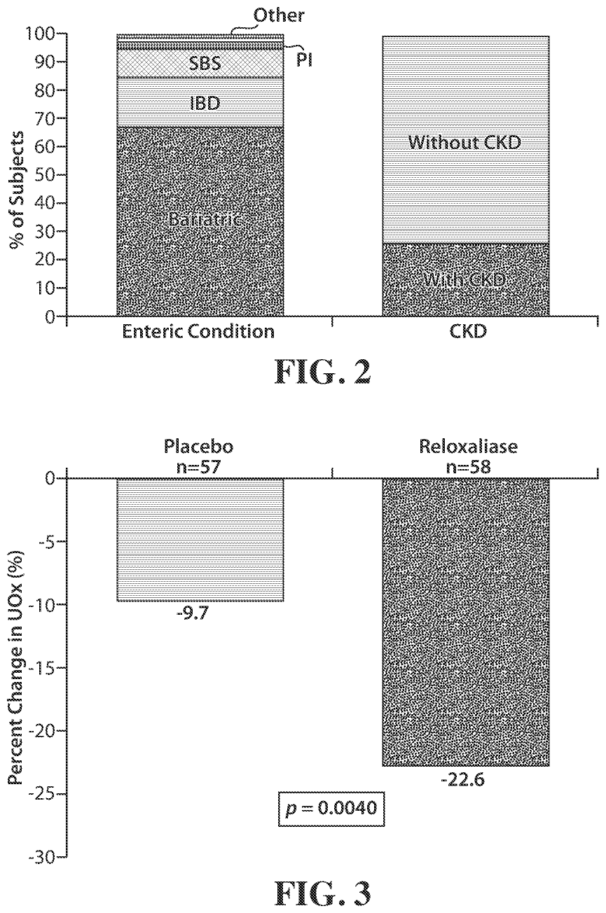

The most common underlying enteric condition was bariatric surgery (68%) followed by IBD (17%). The distribution of underlying enteric conditions is shown in FIG. 2. Overall, mean (SD) UOx at baseline was 89.2 (35.7) mg/day, and 39% had UOx≥90 mg/day. The mean and median number of kidney stones historically reported by subjects in the past 5 years was 11.4 and 3.0, respectively. During the study, 19 subjects had 32 KS events.

The study drug dosing and compliance are shown in TABLE 2. Overall treatment compliance was 97%.

TABLE 2

| | OXDC Crystals | | Placebo | |
|---|---|---|---|---|
| Category/Statistic | (N = 58) | | (N = 57) | |
| Meals/Snacks per day- Mean (SD) | 4.08 | (0.807) | 4.09 | (0.822) |
| Doses per day - Mean (SD) | 3.93 | (0.694) | 3.87 | (0.589) |
| Compliance (%) - Mean (SD) | 96.66 | (4.42) | 96.71 | (4.96) |
| Compliance category: <80% | 1 | (1.7) | 1 | (1.8) |
| Compliance category: 80-120% | 57 | (98.3) | 56 | (98.2) |

Efficacy results are shown in TABLEs 3-4 and FIG. 3. Subjects treated with OXDC Crystals for 4 weeks had a reduction in 24-hour UOx from a baseline of 87.3 to 71.2 mg/24 h across Weeks 1-4, representing a 22.6% reduction. Subjects on placebo had a reduction from 91.1 to 84.6 mg/24 h, representing a 9.7% reduction. The treatment difference of ˜14.3% was highly statistically significant (p=0.0040).

The proportion of subjects with a ≥20% reduction in 24-hour UOx was 48.3% in the OXDC Crystals group, compared with 31.6% in the placebo group (p=0.0605). In the bariatric surgery subgroup, subjects on OXDC Crystals had a 21.2% reduction in 24-hour UOx, compared with a 6.0% reduction on placebo. The treatment difference was ˜16.2% (p=0.0103).

TABLE 3

| | |
|---|---|
| OXDC | |
| Crystals | Placebo |
| (N = 58) | (N = 57) |

Primary Endpoint- Percent Change in 24-h UOx From Baseline During Weeks 1-4

| | | | | |
|---|---|---|---|---|
| Baseline^{a} - Mean (SD) | 87.3 | (28.87) | 91.1 | (41.64) |
| Overall Weeks 1-4 - Mean (SD) | 71.2 | (25.45) | 84.6 | (35.26) |
| Change from baseline - Mean (SD) | −16.1 | (22.28) | −6.6 | (31.35) |
| Percent change from baseline, LS mean (log scale SE) | −22.6 | (0.04) | −9.7 | (0.04) |

| | |
|---|---|
| Comparison between treatments in percent change from | −14.329 (−22.806, −4.922), |
| baseline^{b}: LS mean relative ratio (95% CI), p-value | p = 0.0040 |

CI, confidence interval;
LS, least squares;
MMRM, mixed model repeated measures;
N’, number of subjects dosed;
SD, standard deviation;
SE, standard error;
UOx, urinary oxalate.
^{a}Baseline is defined as the average of the UOx values derived from the two baseline 24-hour urine collections prior to randomization.
^{b}LS means, CIs, and p-values are based on MMRM model for log percent change with fixed effects of treatment group, week, and treatment group-by-week interaction; log Baseline UOx and eGFR as covariates and stratified for stratification factors (bariatric surgery vs other enteric condition, Baseline UOx < 90 vs > 90 mg/24 h, and use of protein pump inhibitors/H2 blockers vs not) including the interaction term of the three stratification factors.

TABLE 4
Secondary Endpoints

| | |
|---|---|
| OXDC | |
| Crystals | Placebo |
| (N = 58) | (N = 57) |

Secondary Endpoint - Proportion With ≥ 20%
Reduction in 24-h UOx From Baseline During Weeks 1-4

| | | | | |
|---|---|---|---|---|
| n/N’ (%) | 28/58 | (48.3) | 18/57 | (31.6) |

| | |
|---|---|
| Comparison between treatments^{a}: | 2.141 (0.967, 4.743). |
| Odds ratio (95% CI), p-value | p = 0.0605 |

Percent change in 24-h UOx from Baseline during
Weeks 1-4, Bariatric Surgery Subgroup

| | | | | |
|---|---|---|---|---|
| Baseline^{b} - Mean (SD) | 88.8 | (31.28) | 92.6 | (36.53) |
| Overall Weeks 1-4 - Mean (SD) | 72.4 | (27.49) | 89.2 | (38.12) |
| Change from baseline - Mean (SD) | −16.5 | (24.89) | −3.5 | (28.71) |
| Percent change from baseline - LS mean (log scale SE) | −21.2 | (0.05) | −6.0 | (0.05) |

| | |
|---|---|
| Comparison between treatments in percent change from | −16.190 (−26.680, −4.200), |
| baseline^{c}: LS mean relative ratio (95% CI), p-value | p = 0.0103 |

Proportion with ≥ 20% Reduction in 24-h UOx from Baseline during
Weeks 1-4, Bariatric Surgery Subgroup

| | | | | |
|---|---|---|---|---|
| n/N’ (%) | 20/40 | (50.0) | 11/38 | (28.9) |

| | |
|---|---|
| Comparison between treatments^{a}: Odds | 2.891 (1.069, 7.815), |
| ratio (95% CI), p-value | p = 0.0364 |

CI, confidence interval;
LS, least squares;
MMRM, mixed model repeated measures;
N’, number of subjects dosed;
SD, standard deviation;
SE, standard error;
UOx, urinary oxalate.
^{a}Odd ratio, confidence interval, and p-value are from a stratified logistic regression model with treatment as the main effect and log baseline UOx and eGFR as covariates, stratified for the stratification factors (bariatric surgery vs other enteric condition, baseline UOx < 90 vs ≥ 90 mg/24 h, and use of proton pump inhibitors/H2 blockers vs not). Expected numbers and percentage of patients for each category are calculated based on parameter estimates.
^{b}Baseline is defined as the average of the UOx values derived from the two baseline 24-hour urine collections prior to randomization.
^{c}LS means, CIs, and p-values are based on MMRM model for log percent change with fixed effects of treatment group, week, and treatment group-by-week interaction; log Baseline UOx and eGFR as covariates and stratified for stratification factors (bariatric surgery vs other enteric condition, Baseline UOx < 90 vs > 90 mg/24 h, and use of protein pump inhibitors/H2 blockers vs not) including the interaction term of the three stratification factors.

OXDC Crystals were generally well tolerated. TEAEs were reported in 69% of OXDC Crystals subjects compared with 52.6% of placebo subjects. TEAEs were considered of mild or moderate intensity, except 1 severe TEAE, which was unrelated to OXDC Crystals. There were no deaths and no related serious adverse events. GI events (abdominal distension, diarrhea, dyspepsia, and flatulence) were reported

### Summary/Conclusion

The phase 3 randomized, placebo-controlled trial (URIROX-1) described in this Example demonstrated that OXDC Crystals led to substantial reduction in measurements of oxalate burden.

In particular, the study achieved its primary endpoint, with a mean reduction of 22.6% in average 24-hour UOx excretion measured during Weeks 1-4 among patients treated with OXDC Crystals, compared to 9.7% in the placebo group least square (LS) mean treatment difference of 14.3%, p=0.004). Additionally, in a pre-specified, stratified analysis of the primary endpoint in bariatric surgery patients (68% of the total study population), patients treated with OXDC Crystals achieved a mean reduction of 21.2% in average 24-hour UOx excretion, compared to 6.0% for patients treated with placebo (LS mean difference of 16.2%, p=0.01).

A secondary endpoint evaluated the proportion of patients on OXDC Crystals with a ≥20% reduction from baseline in 24-hour UOx excretion. Across the full study population, the proportion of patients treated with OXDC Crystals who achieved a ≥20% reduction from baseline in 24-hour UOx excretion was 48.2%, compared to 31.6% for patients on placebo (p=0.06). In the pre-specified, stratified analysis of the key secondary endpoint in bariatric surgery patients, the proportion of patients on OXDC Crystals with a ≥20% reduction from baseline in 24-hour UOx excretion was 50.0%, compared to 28.9% for patients on placebo (p=0.036).

Compliance with the study drug was very high (97%), with no difference between treatment groups. OXDC Crystals were well tolerated. There was a higher proportion of subjects on OXDC Crystals reporting AEs compared with placebo, these were mainly GI in nature.

Together, these results demonstrate that OXDC Crystals degrade oxalate in subjects and suggest that OXDC Crystals have the potential to provide a significant reduction of the oxalate burden on the kidney, a key outcome for patients with EH.

### Example 2

This example describes a clinical study that enrolled subjects with severe enteric hyperoxaluria (EH) with chronic kidney disease (CKD) and hyperoxalemia (elevated plasma oxalate (POx)) to examine the potential of OXDC Crystals to reduce both urine oxalate (UOx) and POx.

### Background

Enteric hyperoxaluria (EH) is a serious metabolic disorder that affects approximately 250,000 people in the United States. EH is characterized by excessive urine oxalate (UOx) excretion that is a complication of increased oxalate absorption due to an underlying gastrointestinal (GI) condition associated with malabsorption (eg, bariatric surgery, short-bowel syndrome [SBS], inflammatory bowel disease [IBD]). Chronically elevated UOx is a major risk factor for progression of kidney stone (KS) disease. KS and inflammation due to oxalate crystal deposition cause permanent damage to the renal parenchyma, which can lead to chronic kidney disease (CKD) and end-stage renal disease (ESRD). Recurrent kidney stones and progressive medullary nephrocalcinosis can contribute to loss of kidney function. With decreasing kidney function, plasma oxalate (POx) levels can rise, resulting in calcium oxalate deposition in the kidneys (oxalate nephropathy) and other tissues and organs, a condition called systemic oxalosis. NALP3-mediated inflammation may be a contributing mechanism for the renal damage in oxalate nephropathy. The presence of oxalate on renal transplant biopsy has been associated with delayed graft function and reduced graft survival. In a recent review of biopsy-proven cases of oxalate nephropathy, ≥50% of subjects required dialysis and most remained dialysis-dependent, with a 33% mortality rate.

The current management for EH includes strict dietary restrictions (low oxalate, low fat, high calcium and fluid intake), with varying degrees of success. With declining kidney function, the GI tract could potentially play a significant role in reducing systemic oxalate burden by serving as an auxiliary kidney.

OXDC Crystals are an oral, non-absorbed, oxalate-specific enzyme therapy that rapidly degrades oxalate in the GI tract. By degrading oxalate along the gut, OXDC Crystals may be able to reduce systemic oxalate burden, KS formation, and further deterioration of kidney function.

### Study Design

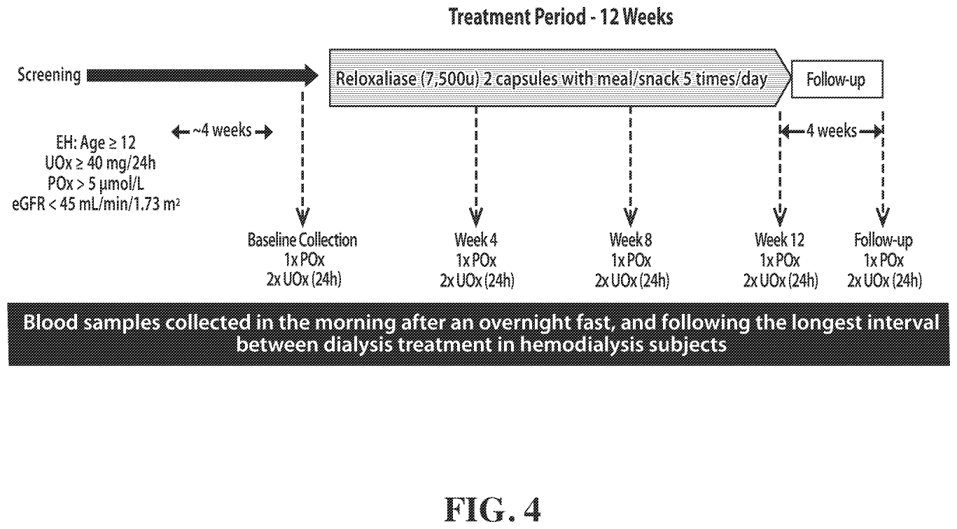

The study was a multi-center, open-label, single-arm Phase II study designed to enroll between 15 and 20 subjects in the United States and Europe aged 12 and older. The general study design is depicted in FIG. 4. Subjects consumed orally 7,500 units of OXDC Crystals with each meal or snack five times a day, for 12 consecutive weeks.

For eligible subjects, one plasma and two 24-hr urine samples were collected for baseline UOx and POx determination. 24-hr urine samples were not obtained in subjects on dialysis or with estimated glomerular filtration rate (eGFR)≤15 mL/min/1.73 m² (CKD stage 5).

The primary efficacy endpoints of the trial were change from baseline in POx, and change from baseline in 24-hr UOx excretion (normalized to creatinine). The mean change from baseline in POx and/or UOx compared to the average change across the 12 weeks of treatment was calculated for subjects who had at least 2 of the 3 time points available. In addition, the mean maximal change from baseline was calculated. Safety assessments were treatment-emergent adverse events (TEAEs) and routine safety laboratory tests.

Key inclusion criteria were history of EH with UOx at screening ≥40 mg/24 h from an adequate collection (i.e., appropriate ratio of creatinine [mg]/body weight [kg] for gender), and an eGFR <45 mL/min/1.73 m² and POx>5 μmol/L at screening.

Key exclusion criteria were unable or unwilling to discontinue vitamin C supplementation, and active autoimmune disorder or other condition requiring high doses of systemic steroids (e.g. >10 mg/day prednisone or equivalent) or intensification of immunosuppressant therapy ≤1 month prior to/during screening.

### Results

Of the 9 EH subjects who were enrolled, 6 (67%) completed the study, 2 are ongoing, and 1 subject discontinued the study drug prematurely and withdrew consent. All 9 EH subjects were Caucasian, with a median (min, max) age of 66 (55, 76) years. The majority of subjects were male (n=6; 67%), 5 were on dialysis, and 3 were post-kidney transplant.

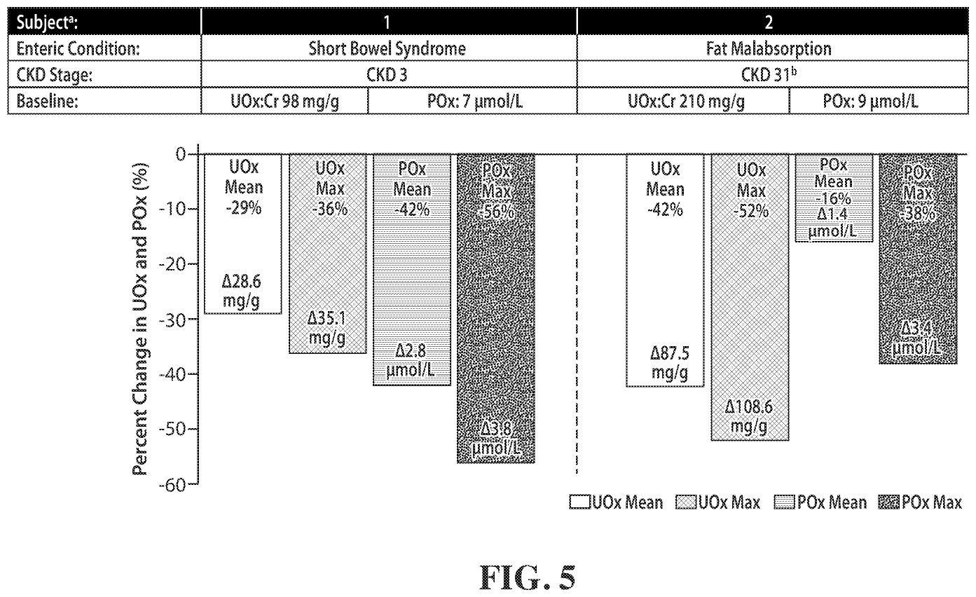

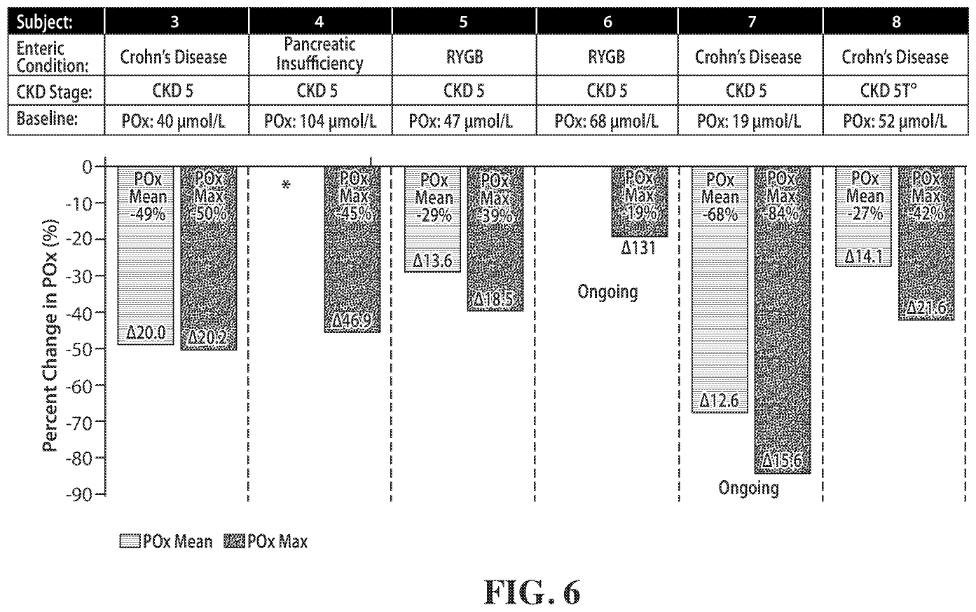

Results for subjects with CKD stage 3b (n=2) are depicted in FIG. 5. Results for subjects with CKD stage 5 (dialysis or eGFR <15 mL/min/1.73 m²) (n=6) are depicted in FIG. 6. The mean reduction from baseline of POx was 38.5% (˜68% to ˜16%) in all subjects. In subjects with CKD stage 5, mean reduction was 43.3% (−67% to ˜27%). In 2 subjects with CKD stage 3, OXDC Crystals reduced 24-hour UOx from baseline by 29% and 42%, with maximal reduction by 36% and 52%, respectively.

OXDC Crystals were generally well tolerated. A summary of adverse events is shown in TABLE 5. No drug-related serious adverse events (SAEs) were reported. TEAEs were most commonly reported in the GI system organ class (n=6; 66.7%). One subject experienced a TEAE not related to study drug as determined by the investigator. The subject subsequently decided to discontinue treatment, but completed study visits. Two subjects discontinued study drug early. One subject experienced a TEAE (not related to study drug); the subject subsequently decided to discontinue treatment after the week 8 visit. One subject had a drug interruption due to GI adverse event (AE), which did not recur on re-challenge. The subject subsequently withdrew consent.

TABLE 5

| Category | Subjects (n = 9) | | Events (n = 31) | |
|---|---|---|---|---|
| TEAE^{a}, n (%) | 8 | (88.9) | 31 | (100) |
| Any treatment- | 2 | (22.2) | 2 | (6.5) |
| emergent SAE, n (%) | | | | |
| GI TEAE, n (%) | 6 | (66.7) | 10 | (32.3) |
| Related GI TEAE, n (%) | 2^{b} | (22.2) | 5 | (16.1) |
| Abdominal distention | 1 | (11.1) | 1 | (3.2) |
| Diarrhea | 1 | (11.1) | 1 | (3.2) |
| Flatulence | 1 | (11.1) | 3 | (9.7) |

^{a}TEAE defined as AE with onset at the time of or following the first dose of treatment with study drug through 12 weeks after their last dose of study medication
^{a}One subject had 2 related GI TEAEs.

### Summary/Conclusions

The Phase H studied described in this Example demonstrates the effect of OXDC Crystals in reducing POx levels in subjects with EH and CKD with hyperoxalemia. OXDC Crystals decreased POx in subjects with a variety of underlying conditions associated with EH, including Crohn's disease, bariatric surgery, pancreatic insufficiency, short bowel syndrome, and fat malabsorption. Two patients with CKD Stage 3 demonstrated a substantial reduction in 24-hour UOx excretion over weeks 4 to 12 (mean reduction 29% and 42%). These patients also showed a significant reduction in plasma oxalate (POx) (mean reduction 42% and 16%, respectively). Six patients with CKD Stage 5, including five patients on dialysis, demonstrated meaningful reductions in POx levels over weeks 4 to 12 (mean reduction 43.3%, ranged from 27% to 68%). Oral therapy with OXDC Crystals for 12 weeks was generally well tolerated.

Together, these results demonstrate a pharmacological strategy for POx reduction in subjects with EH.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of treating a subject with enteric hyperoxaluria, the method comprising orally administering to the subject an effective amount of biologically active oxalate decarboxylase (OXDC) crystals up to 5 times per day; wherein the level of 24-hour urinary oxalate (UOx) excretion of the subject is reduced by at least 20% relative to the level of 24-hour UOx excretion prior to treatment, wherein the subject (i) is receiving a proton pump inhibitor and/or an acid blocker, and/or (ii) is at risk of developing advanced chronic kidney disease (CKD).

2. The method of claim 1, wherein the subject (i) has had bariatric surgery, (ii) is receiving a proton pump inhibitor and/or an acid blocker, or (iii) has had bariatric surgery and is receiving a proton pump inhibitor and/or an acid blocker.

3. A method of treating a subject with enteric hyperoxaluria, the method comprising administering a dosing regimen of biologically active oxalate decarboxylase (OXDC) crystals to the subject, wherein, when the dosing regimen is administered to subjects with enteric hyperoxaluria, the dosing regimen causes (i) at least about a 20% mean reduction in the baseline level of 24-hour UOx excretion, and (ii) a reduction in kidney stone disease progression in at least about 5%, 10%, 15%, 20%, 25%, 30%, 35%, or 40% of the subjects and/or a reduction in kidney stone disease progression in a proportion of subjects that is at least about 5%, 10%, 15%, 20%, 25%, 30%, 35%, or 40% greater than the proportion of untreated subjects with a reduction in kidney stone disease progression.

4. A method of treating a subject with enteric hyperoxaluria, the method comprising orally administering to the subject an effective amount of biologically active oxalate decarboxylase (OXDC) crystals up to 5 times per day; wherein the subject (i) is receiving a proton pump inhibitor and/or an acid blocker, and optionally (ii) has had bariatric surgery.

5. The method of claim 4, wherein the level of 24-hour UOx excretion of the subject is reduced by at least 20% relative to the level of 24-hour UOx excretion prior to treatment.

6. The method of any one of claims 1-5, wherein the OXDC crystals are administered every day for at least 28 days.

7. The method of claim 6, wherein the OXDC crystals reduce 24-hour UOx excretion within 7 days after the initial administration of the OXDC crystals.

8. A method of treating a subject with enteric hyperoxaluria and advanced chronic kidney disease (CKD), the method comprising orally administering to the subject an effective amount of biologically active oxalate decarboxylase (OXDC) crystals up to 5 times per day, whereupon administration of the OXDC crystals causes,
(a) a reduction in the level of 24-hour urinary oxalate (UOx) excretion of the subject by 25-50% relative to the level of 24-hour UOx excretion prior to treatment; and/or
(b) a reduction in the plasma oxalate (POx) level of the subject by 15-80% relative to the level of POx prior to treatment.

9. The method of claim 8, wherein the subject in need thereof has (i) UOx excretion of >40 mg/24 hours (normalized for creatinine level), (ii) plasma oxalate (POx) level of >5 μmol/L, and/or (iii) eGFR >45 mL/min/1.73 m².

10. A method of treating a subject with enteric hyperoxaluria, the method comprising administering a dosing regimen of biologically active oxalate decarboxylase (OXDC) crystals to the subject, wherein, when the dosing regimen is administered to subjects having enteric hyperoxaluria and advanced chronic kidney disease (CKD), the dosing regimen causes (a) about a 25-50% mean reduction in the baseline level of 24-hour UOx excretion; and/or (b) about a 15-80% mean reduction in the baseline level of plasma oxalate (POx) level.

11. The method of any one of claims 1-2 and 4-10, wherein, when the dosing regimen is administered to such subjects, the dosing regimen causes a reduction in kidney stone disease progression in at least about 5%, 10%, 15%, 20%, 25%, 30%, 35%, or 40% of the subjects and/or a reduction in kidney stone disease progression in a proportion of subjects that is at least about 5%, 10%, 15%, 20%, 25%, 30%, 35%, or 40% greater than the proportion of untreated subjects with a reduction in kidney stone disease progression.

12. A method of treating a subject with enteric hyperoxaluria and advanced chronic kidney disease (CKD), the method comprising orally administering to the subject an effective amount of biologically active oxalate decarboxylase (OXDC) crystals up to 5 times per day; wherein the subject in need thereof has (i) UOx excretion of >40 mg/24 hours (normalized for creatinine level), (ii) plasma oxalate (POx) level of >5 μmol/L, and/or (iii) eGFR >45 mL/min/1.73 m².

13. The method of claim 12, wherein administration of the OXDC crystals causes a reduction in the level of 24-hour urinary oxalate (UOx) excretion of the subject by 25-50% relative to the level of 24-hour UOx excretion prior to treatment.

14. The method of claim 12 or 13, wherein administration of the OXDC crystals causes a reduction in the plasma oxalate (POx) level of the subject by 15-80% relative to the level of POx prior to treatment.

15. The method of any one of claims 1-14, wherein the subject has had bariatric surgery.

16. The method of claim 15, wherein the level of 24-hour urinary oxalate (UOx) excretion of the subject is reduced by at least 10%, 20%, or 30% relative to the level of 24-hour UOx excretion prior to treatment.

17. The method of claim 16, wherein the OXDC crystals are administered to the subject for at least 24 weeks, and during weeks 1-4 and 16-24 that the OXDC crystals are administered to the subject, the level of 24-hour urinary oxalate (UOx) excretion of the subject is reduced by at least 10%, 20%, or 30% relative to the level of 24-hour UOx excretion prior to treatment.

18. A method of treating a subject with enteric hyperoxaluria who has had bariatric surgery, the method comprising administering a dosing regimen of biologically active oxalate decarboxylase (OXDC) crystals to the subject, wherein, when the dosing regimen is administered to subjects with enteric hyperoxaluria who have had bariatric surgery, the dosing regimen causes in at least 10%, 20%, 30%, 40%, or 50% of the subjects a reduction of at least 20% in the level of 24-hour UOx excretion relative to prior to treatment.

19. The method of claim 18, wherein the dosing regimen is administered to the subject for at least 4 weeks, and during weeks 1-4 that the dosing regimen is administered to the subject, the dosing regimen causes in at least 10%, 20%, 30%, 40%, or 50% of the subjects a reduction of at least 20% in the level of 24-hour UOx excretion relative to prior to treatment.

20. The method of claim 18 or 19, wherein, when the dosing regimen is administered to subjects with enteric hyperoxaluria who have had bariatric surgery, the dosing regimen causes (i) at least about a 20% mean reduction in the baseline level of 24-hour UOx excretion, and/or (ii) a reduction in kidney stone disease progression in at least about 5%, 10%, 15%, 20%, 25%, 30%, 35%, or 40% of the subjects and/or a reduction in kidney stone disease progression in a proportion of subjects that is at least about 5%, 10%, 15%, 20%, 25%, 30%, 35%, or 40% greater than the proportion of untreated subjects with a reduction in kidney stone disease progression.

21. The method of any one of claims 1-20, wherein the OXDC crystals are administered every day for at least 12 consecutive weeks.

22. The method of claim 21, wherein the OXDC crystals are administered every day for from about 16 to about 24 consecutive weeks.

23. The method of any one of claims 1-22, wherein the OXDC crystals are administered every day for at least 16, 20, 24, 28, 32, 36, 40, 44, or 48 consecutive weeks, or 12, 16, 20, 24, 36, 48, 52, 54, or 60 consecutive months.

24. The method of any one of claims 1-23, wherein the subject has stage 3 CKD or stage 5 CKD.

25. The method of claim 24, wherein the 24-hour UOx excretion of a stage 3 CKD subject is reduced within 4 to 12 weeks after initiating treatment by 25-45%, relative to the level of 24-hour UOx excretion prior to treatment.

26. The method of claim 24, wherein the POx level of a stage 3 CKD subject is reduced within 4 to 12 weeks after initiating treatment by 15-45% relative to the level of POx prior to treatment.

27. The method of claim 24, wherein the POx level of a stage 5 CKD subject is reduced within 4 to 12 weeks after initiating treatment by 25-70% relative to the level of POx prior to treatment.

28. The method of any one of claims 1-27, wherein the OXDC crystals are administered with a food (e.g., a meal or a snack).

29. The method of any one of claims 1-28, wherein two dosage units each comprising 3,750 units of OXDC crystals are administered up to 5 times per day.

30. The method of any one of claims 1-28, about 284 mg of OXDC crystals are administered up to 5 times per day.

31. The method of any one of claims 1-30, wherein 120-150 mg of OXDC crystals are formulated in a capsule for oral administration.

32. The method of any one of claims 1-31, wherein the subject is a pediatric subject.

33. The method of any one of claims 1-32, wherein urine supersaturation of calcium oxalate in the subject is reduced relative to prior to treatment.

34. The method of claim 33, wherein urine supersaturation of calcium oxalate in the subject is reduced by at least 5%, 10%, 15%, 20%, 25%, 30%, 35%, 40%, 45%, or 50% relative to prior to treatment.

35. The method of any one of claims 1-34, wherein eGFR in the subject is reduced relative to prior to treatment.

36. The method of claim 35, wherein eGFR in the subject is reduced by at least 15%, 20%, 25%, 30%, 35%, 40%, or 45% relative to prior to treatment.

37. The method of claim 36, wherein eGFR in the subject is reduced by at least 30% relative to prior to treatment.

38. A method of treating subjects with enteric hyperoxaluria, the method comprising orally administering to the subjects an effective amount of biologically active oxalate decarboxylase (OXDC) crystals up to 5 times per day, wherein when the dosing regimen is administered to subjects, the dosing regimen causes: (a) at least about a 20% mean reduction in the baseline level of 24-hour UOx excretion; (b) about a 25-50% mean reduction in the baseline level of 24-hour UOx excretion; (c) about a 15-80% mean reduction in the baseline level of plasma oxalate (POx) level; (d) in at least 10%, 20%, 30%, 40%, or 50% of the subjects a reduction of at least 20% in the level of 24-hour UOx excretion relative to prior to treatment; and/or (e) a reduction in kidney stone disease progression in at least about 5%, 10%, 15%, 20%, 25%, 30%, 35%, or 40% of the subjects and/or a reduction in kidney stone disease progression in a proportion of subjects that is at least about 5%, 10%, 15%, 20%, 25%, 30%, 35%, or 40% greater than the proportion of untreated subjects with a reduction in kidney stone disease progression.

39. The method of claim 38, wherein the subjects: (a) are receiving a proton pump inhibitor and/or an acid blocker, (b) have or are at risk of developing advanced chronic kidney disease (CKD), and/or (c) have had bariatric surgery.
